(19) United States
(12) Patent Application Publication
(10) Pub. No.: US 2009/0131524 A1
(43) Pub. Date: May 21, 2009

(54) SIALIC ACID ABC TRANSPORTERS IN PROKARYOTES THERAPEUTIC TARGETS

(75) Inventors: Bradford W. Gibson, Berkeley, CA (US); Robert S. Munson, Hilliard, OH (US); Deborah M. Post, Fairfax, CA (US)

Correspondence Address: Weaver Austin Villeneuve & Sampson LLP, P.O. BOX 70250, OAKLAND, CA, 94612-0250, US

(73) Assignee: BUCK INSTITUTE, Novato, CA (US)

(21) Appl. No.: 11/916,975

(22) PCT Filed: May 31, 2006
(86) PCT No.: PCT/US2006/021202

§ 371 Date: Dec. 22, 2008

Related U.S. Application Data
(60) Provisional application No. 60/689,151, filed on Jun. 7, 2005.

(51) Int. Cl. A61K 31/195; C12N 1/20; C12N 15/11; C12N 15/00; C12N 1/21; C07K 14/00; C12N 5/06; C07K 16/18; G01N 33/569; C12Q 1/02

(52) U.S. Cl. 514/561; 435/252.1; 435/253.3; 435/252.8; 435/253.5; 536/23.1; 435/320.1; 435/252.3; 530/350; 435/325; 530/387.9; 435/7.8; 435/29

(57) ABSTRACT

This invention provides a novel bacterial sialic acid transporter that is a member of the family of ABC transporters. The transporter is a useful target for pharmaceuticals.

### CROSS-REFERENCE TO RELATED APPLICATIONS

This application claims benefit of and priority to U.S. Ser. No. 60/689,151, filed on Jun. 7, 2005, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in part, by Grant Nos: K12 HD43 372 and AI3 1254 from the National Institutes of Health. The Government of the United States of America has certain rights in this invention.

### FIELD OF THE INVENTION

This invention pertains to the field of bacteriology and molecular biology. In particular this invention pertains to the discovery and characterization of a bacterial sialic acid transporter that is a member of the ABC transporter family.

### BACKGROUND OF THE INVENTION

Haemophilus ducreyi is the causative agent of the sexually transmitted disease chancroid. This disease is most prevalent in developing countries (54); however, sporadic outbreaks occur in predominantly urban areas of the United States (62). Chancroid has been linked, as a cofactor, to the heterosexual transmission of the human immunodeficiency virus (KIV), especially in areas where both diseases are predominant (28, 54, 61, 62).

A number of putative virulence factors of H. ducreyi have been described which seem to play a role in pathogenicity. Two of these factors are toxins: a hemolytic toxin (3, 42, 71) and cytolethal distending toxin (10, 11, 14). The outer membrane proteins, DsrA and DltA, have been shown to promote resistance to killing by normal human serum (17, 34). The hemoglobin receptor, HgbA (16, 56), and the Cu,Zn-superoxide dismutase (33, 41, 49) both seem to play a role in iron acquisition for H. ducreyi. Additionally, a number of proteins have been shown to play a role in adherence (9, 19, 34).

The lipooligosaccharide (LOS) produced by H. ducreyi is also a putative virulence factor. Previous studies have shown that LOS plays a role in adherence of bacteria to keratinocytes and human foreskin fibroblasts (2, 20). Structural studies have been performed on the LOS from a number of H. ducreyi strains (1, 6, 7, 20, 21, 39, 40, 51, 52). These studies have shown that one of the predominant glycoforms expressed by H. ducreyi terminates in N-acetyllactosamine. These same terminal sugars are also found on LOS structures expressed by Neisseria meningitidis, Neisseria gonorrhoeae, and Haemophilus influenzae (reviewed in (47)). Previous findings have shown that some of the LOS structures from these bacteria mimic human antigens (35, 36) (reviewed in (26, 63) and are involved in receptor-mediated interactions (24, 25, 58, 59).

The LOS glycoform terminating in N-acetyllactosamine can be further extended by the addition of a single sialic acid (N-acetyl-neuraminic acid, NeuAc) to the terminal galactose residue. Studies have shown that sialic acid is an important virulence factor in N. gonorrhoeae and N. meningitidis, promoting resistance to bactericidal activity of normal human serum (5, 29, 43, 70). Additionally, sialylation of N. meningitidis LOS has been shown to increase resistance to phagocytosis by human dendritic cells (65). Previous studies from our laboratory have shown that the LOS of most H. ducreyi strains, including the prototype strain 35000HP, are highly sialylated (39), but the mechanism of sialic acid transport has been unclear.

### SUMMARY OF THE INVENTION

This invention pertains to the discovery of a sialic acid transporter that is a member of the family of ABC transporters. The sialic acid ABC transporter provides a good target for screening for drugs that inhibit the growth and/or proliferation and/or infectivity of a bacterium expressing such a transporter. In addition, agents that inhibit expression and/or activity of the transporter can inhibit growth and/or proliferation and/or infectivity of the bacterium.

Thus, in certain embodiments, this invention provides methods of treating a mammal infected with a bacterium by administering a bacterial sialic ABC transporter inhibitory agent, e.g., wherein the inhibitor is administered in an amount that reduces the uptake of sialic acid by the bacterium. An illustrative mammal to be treated is a human although various veterinary uses are also contemplated. Typically the infecting bacterium is a pathogenic organism containing a sialic acid ABC transporter with high homology to the H. ducreyi transporter described herein and has sialic acid as a part of the cell wall structure. Illustrative organisms include, but are not limited to Haemophilus ducreyi, Haemophilus influenzae, Haemophilus somnus, Haemophilus gallarium, Vibrio vulnificus, Vibrio cholera, Shigella flexneri, Pseudomonas aeruginosa, Helicobacter pylori, Pasturella multicidia, Salmonella enteritidis, Actinobacillus pleuropneunoniae. Haemophilus somnus, Corynebacterium glutamicum, Corynebacterium diphtheriae, and Streptomyces avermitilis. In certain situations, the mammal to be treated may be infected with a combination of bacterial strains.

In various embodiments the inhibitory agent can be a viral neuraminidase inhibitor, such as 3-fluoro-N-acetylneuarmainic acid (3FNA), N-acetyl-2,3-didehydro-2-deoxyneuraminic acid (DDNA), the 4-guanidino-derivative of DDNA (Relenza®) or Tamiflu®. Alternatively, or additionally, the inhibitory agent may be an N-alkanoyl-derivative of sialic acid, such a 5-N-octanoyl derivative of sialic acid (SiaOct).

In various embodiments in the method of the present invention the uptake of sialic acid by the bacterium is reduced by at least 10%. It can be reduced by 20%, 30%, 40%, 50%, 60%, 70%, 80%, 90%, 95%, 96%, 97%, 98%, 99%, or even 100%.

In certain embodiments the present invention also provides isolated/purified polypeptide(s) that are components of a bacterial sialic acid ABC transporter such as an ABC transporter derived from Haemophilus ducreyi, Haemophilus influenzae, Haemophilus somnus, Haemophilus gallarium, Vibrio vulnificus, Vibrio cholera, Shigella flexneri, Pseudomonas aeruginosa, Helicobacter pylori, Pasturella multicidia, Salmonella enteritidis, Actinobacillus pleuropneumoniae. Haemophilus somnus, Corynebacterium glutamicum, Corynebacterium diphtheriae, Streptomyces avermitilis, and the like.

Also provided are antibodies that specifically bind a polypeptide(s) comprising components of a bacterial sialic acid ABC transporter protein.

In certain embodiments the proteins, nucleic acids, transporters and cells expressing proteins and/or transporters specifically exclude those in which the protein/nuclei acid is the Escherichia coli NanT protein/nucleic acid.

The details of one or more embodiments of the invention are set forth in the accompanying drawings and the description below. Other features, objects, and advantages of the invention will be apparent from the description and drawings, and from the claims.

### DEFINITIONS

ABC transporters typically contain two membrane-spanning domains that harbor a translocation pathway for a specific substrate. Attached are two cytoplasmic adenosine triphosphate-binding cassettes (hence ABC). As the ABC cassettes bind and hydrolyze ATP, conformational changes occur that are transmitted to the membrane-spanning domains, where they induce rearrangements that translocate the substrate from one side of the membrane to the other. The initial motion of the ABC cassettes has been dubbed the power stroke, and it is generally assumed that this rearrangement is similar in all ABC transporters, irrespective of the size of the substrate to be transported or the directionality of the translocation (import or export).

“Sialic acid ABC transporter proteins” are proteins that comprise a sialic acid ABC transporter.

The terms “isolated”, “purified”, or “biologically pure” when referring to an isolated polypeptide refer to material that is substantially or essentially free from components that normally accompany it as found in its native state. With respect to nucleic acids and/or polypeptides the term can refer to nucleic acids or polypeptides that are no longer flanked by the sequences typically flanking them in nature. Chemically synthesized polypeptides are “isolated” because they are not found in a native state (e.g. in blood, serum, etc.). In certain embodiments, the term isolated indicates that the polypeptide is not found in nature.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence of a biomolecule in a heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all, to other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions, e.g., containing formamide (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331).

The term “specifically binds”, as used herein, when referring to a biomolecule (e.g., protein, nucleic acid, antibody, etc.), refers to a binding reaction that is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the complementary sequence is identical to all or a portion of a reference polynucleotide sequence.

Sequence comparisons between two (or more) polynucleotides or polypeptides are typically performed by comparing sequences of the two sequences over a “comparison window” to identify and compare local regions of sequence similarity. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are optimally aligned.

Optimal alignment of sequences for comparison may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms such as CLUSTALW, GAP, BESTFIT, BLAST, FASTA, and TFASTA (Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 60% sequence identity, preferably at least 80%, more preferably at least 90% and most preferably at least 95%, compared to a reference sequence using the programs described above (preferably BLAST) using standard parameters. In one embodiment, 25% sequence identity over a window of 200 amino acids coupled with information regarding the apophytochrome consensus sequence is sufficient to identify a new apophytochrome. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%, preferably at least 60%, more preferably at least 90%, and most preferably at least 95%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Another indication that nucleotide sequences are substantially identical is if two nucleic acid molecules hybridize to each other, or to a third nucleic acid, under stringent conditions. Stringent conditions are sequence dependent and will be different in different circumstances. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched complementary nucleic acid sequence. Typically, stringent conditions will be those in which the salt concentration is about 0.02 molar at pH 7 and the temperature is at least about 60° C. Stringent conditions for a standard Southern hybridization will include at least one wash (usually 2) in 0.2×SSC at a temperature of at least about 50° C., usually about 55° C., for 20 minutes, or equivalent conditions.

The terms “high homology” or “high identity” are used interchangeable to refer to sequence identity. Proteins having high identity at the amino acid level have at least 50%, preferably at least 60 or 70%, more preferably 1 at least 80 or 90%, and most preferably at least 95, or 98% sequence identity.

The term “conservative substitution” is used in reference to proteins or peptides to reflect amino acid substitutions that do not substantially alter the activity (specificity or binding affinity) of the molecule. Typically conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### BRIEF DESCRIPTION OF THE DRAWINGS

FIGS. 1A, 1B, and 1C illustrate LOS structure, nomenclature, and SDS-PAGE. FIG. 1A: H. ducreyi strain 35000HP LOS structure and nomenclature (6).

FIG. 1B: Silver stained SDS-PAGE of LOS isolated from 35000HP (lanes 1 & 10), 35000HP-305, 35000HP-306, 35000HP-310, 35000HP-313, 35000HP-319, 35000HP-320, 35000HB-322, 1st mutant 35000HP-RSM203 (lanes 2-9, respectively). The sialylated glycoform (A₅a₁) is absent from all of the LOS samples except the parent strain 35000HP (indicated with an arrow). In the absence of the A₅a₁ structure the b branch structures (A₅B₁ and A₅b₂) are more readily observed. FIG. 1C: Silver stained SDS-PAGE of LOS isolated from 35000HP (lanes 1 & 2), 1st mutant 35000HP-RSM203 (lanes 3 & 4), and 35000HP-310 (lanes 5 & 6). Lanes designated with a + are LOS samples which were treated with neuraminidase, lanes designated with a − are LOS samples which were not treated with neuraminidase. The neuraminidase treatment only affected the glycoforms present in the 35000HP LOS (sialylated glycoform indicated with an arrow); this treatment had no affect on the LOS from the 1st mutant 35000HP-RSM203 or 35000HP-310. These data are representative of all the sialic acid transporter mutants presented in this study. The three core heptoses (Hep) are L-glycero-D-manno-heptose, the branch heptose, in italics, is of the D-glycero-D-manno configuration.

FIGS. 2A and 2B show MALDI-TOF spectra of O-deacylated LOS isolated from 35000HP and 35000HP-306 grown on (FIG. 2A) chocolate agar plates and (FIG. 2B) chocolate agar plates supplemented with 1 mM sialic acid. The spectra from the two strains are quite similar, except for the absence of the sialylated glycoform peaks, A₅a₁ and A₅a_{1*}, in the 35000HP-306 spectra. Additionally, the A₅b₁, A₅b₁ and A₅b₂ peaks, corresponding to the addition of GlcNAc (without and with PEA) and lactosamine to the A₅ structure, respectively, seem more prevalent in the 35000HP-306 spectra. Corresponding with previous findings (50), the sialylated glycoform peak A₅a₁* is more abundant in the 35000HP sample grown on sialic acid supplemented media. The 35000HP-306 data are representative of all the sialic acid transporter mutants presented in this study.

FIG. 3 illustrates an open reading frame (ORF) map of the H. ducreyi sialic acid transporter (sat) region. The genes satAB CD, corresponding to Hd1669-1672, respectfully, and their direction of transcription are shown. The site of transposon insertion in each mutant is indicated with the corresponding mutant strain name.

FIGS. 4A and 4B illustrate the results of a sialic acid uptake assay. Bacteria were grown on plates and suspended to an OD₆₀₀ₙₘ of 2. Bacteria were then incubated with [³H]-NeuAc for the indicated time, filtered, washed, and counted. FIG. 4A: A 15 min time course study was performed on the parent strain 35000HP. The plot is the mean of triplicate determinations. FIG. 4B: A single 10 min time point determination of [³H]-NeuAc uptake from the satB mutant 35000HP-306 (▪), from the satD mutant 35000HP-313 [symbol] from the satA mutant 35000HP-319 [symbol] from the satC mutant 35000HP-322 [symbol] and from the parent strain 35000HP [symbol] The results are the mean of triplicate determinations. Results shown are representative of at least two independent experiments.

FIG. 5, panels A-C, illustrate a hypothetical model for sialic acid transport in H. ducreyi. Protein functions for the model were based on sequence comparisons with other ABC-transporter systems. Panel A: SatA in a closed conformation, with sialic acid bound, interacts with SatB and SatC to initiate transport and hydrolysis. Panel B: When SatA binds tightly with SatB/SatC it may transition to an open conformation which has decreased affinity for sialic acid, and SatB/SatC may reorient to expose a sugar-binding site. Panel C: When ATP is hydrolyzed, sialic acid is transported, SatA is released, and SatB/SatC return to their original conformation.

### DETAILED DESCRIPTION

This invention pertains to the identification of a novel transporter of sialic acid in the human pathogen, Haemophilus ducreyi. H. ducreyi is the causative agent of chancroid, a sexually transmitted disease in humans. A major surface antigen in H. ducreyi is a lipooligosaccharide (LOS), which is partially modified by sialic acid at a terminal N-acetyllactosamine disaccharide (or lactosamine) to form LOS now terminating in sialyl-N-acetyllactosamine. H. ducreyi does not synthesize sialic acid, which must be acquired from the host during infection or from the culture medium when the bacteria are grown in vitro. A library of random transposon mutants in H. ducreyi were screened for mutants that were unable to add sialic acid to N-acetyllactosamine-containing LOS. Mutants that reacted with the monoclonal antibody 3F11, which recognizes the terminal lactosamine structure of LOS, and lacked reactivity with the lectin Maackia amurensis agglutinin (MAA), which recognizes α2,3-linked sialic acid, were further characterized to demonstrate that they produced a N-acetyllactosamine-containing LOS by silver stained SDS-PAGE and mass spectrometric analyses. The genes interrupted in these mutants were mapped to a four gene cluster, satA-D, with similarity to genes encoding bacterial ABC transporters.

Uptake assays using radiolabeled sialic acid confirmed that the mutants were unable to transport sialic acid. This is the first report of bacteria using an ABC transporter for sialic acid uptake, although based on gene similarity, it is likely that other organisms or pathogens use a similar ABC-type transporter for sialic acid (e.g., H. somnus).

Using the information provided herein, other sialic acid ABC transporters can be readily identified simply by searching, e.g. bacterial genome databases, for nucleic acid and/or proteins substantial sequence identity to the sialic acid ALBC transporters identified herein.

In addition, the components of the sialic acid ABC transporters (e.g. the periplamic-binding domain of the ABC transporter, SatA, whose presumed function is to bind sialic acid. SatA, or other protein components of the transporter (i.e., SatB, SatC and SatD)) can provide good targets for screening for therapeutic agents, e.g. sialic acid analogs, that bind to and/or inhibit the expression or activity of the transporter. Such compounds can be used as anti-microbials by inhibiting a key step (i.e., sialic acid uptake) that may be required for an organism to successfully invade or survive in the host (animal or human). Alternatively, they can serve as antigen targets for immune therapy. Alternatively, in certain embodiments they can serve as antigen targets for immune therapy.

This invention also contemplates the preparation of isolated sialic acid ABC transporter proteins, the preparation of antibodies (e.g., polyclonal, monoclonal, single chain, etc.) that bind such, the preparation of cells that express heterologous sialic acid ABC transporter proteins, and the like.

### I. Cloning and Expression of Sialic Acid ABC Transporter Proteins.

It is often desirable to provide isolated sialic acid ABC transporter polyepeptides. These polypeptides can be used to raise an immune response and thereby generate antibodies specific to the intact sialic acid ABC transporter protein or to various subsequences or domains thereof, or in the formulatin of vaccines to induce an immune response against pathogens expressing a sialic acid ABC transporter. As explained below, sialic acid ABC transporter protein polypeptides and various fragments thereof can be conveniently produced using synthetic chemical syntheses or recombinant expression methodologies. In addition to the intact full-length sialic acid ABC transporter protein polypeptide(s), in some embodiments, it is often desirably to express immunogenically relevant fragments (e.g. fragments that can be used to raise specific anti-sialic acid ABC transporter protein antibodies).

A) De Novo Chemical Synthesis.

The sialic acid ABC transporter polypeptide(s), or fragments thereof can be synthesized using standard chemical peptide synthesis techniques. Where the desired subsequences are relatively short (e.g., when a particular antigenic determinant is desired) the molecule can be synthesized as a single contiguous polypeptide. Where larger molecules are desired, subsequences can be synthesized separately (in one or more units) and then fused by condensation of the amino terminus of one molecule with the carboxyl terminus of the other molecule thereby forming a peptide bond.

Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. (1963) J. Am. Chem. Soc., 85: 2149-2156, and Stewart et al. (1984) Solid Phase Peptide Synthesis, 2nd ed. Pierce Chem. Co., Rockford, Ill.

B) Recombinant Expression.

In a certain embodiments, the sialic acid ABC transporter proteins or subsequences thereof, are synthesized using recombinant expression systems. Generally this involves creating a DNA sequence that encodes the desired protein, placing the DNA in an expression cassette under the control of a particular promoter, expressing the protein in a host, isolating the expressed protein and, if required, renaturing the protein.

DNA encoding the sialic acid ABC transporter protein proteins described herein (e.g., SEQ ID NOs:1, 2, 3, 4, etc.) can be prepared by any suitable method as described above, including, for example, cloning and restriction of appropriate sequences or direct chemical synthesis.

This nucleic acid can be easily ligated into an appropriate vector containing appropriate expression control sequences (e.g. promoter, enhancer, etc.), and, optionally, containing one or more selectable markers (e.g. antibiotic resistance genes).

The nucleic acid sequences encoding sialic acid ABC transporter protein proteins or protein subsequences can be expressed in a variety of host cells, including, but not limited to, E. coli, other bacterial hosts, yeast, fungus, and various higher eukaryotic cells such as insect cells (e.g. SF3), the COS, CHO and HeLa cells lines and myeloma cell lines. The recombinant protein gene will typically be operably linked to appropriate expression control sequences for each host. For E. coli this can include a promoter such as the T7, trp, or lambda promoters, a ribosome binding site and preferably a transcription termination signal. For eukaryotic cells, the control sequences can include a promoter and often an enhancer (e.g., an enhancer derived from immunoglobulin genes, SV40, cytomegalovirus, etc.), and a polyadenylation sequence, and may include splice donor and acceptor sequences.

The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant sialic acid ABC transporter protein protein(s) can be purified according to standard procedures of the art, including ammonium sulfate precipitation, affinity columns, column chromatography, gel electrophoresis and the like (see, generally, R. Scopes, (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred. Once purified, partially or to homogeneity as desired, the polypeptides may then be used (e.g., as immunogens for antibody production).

One of skill in the art would recognize that after chemical synthesis, biological expression, or purification, the sialic acid ABC transporter protein protein(s) may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (see, e.g., Debinski et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski et al., for example, describes the denaturation and reduction of inclusion body proteins in guanidine-DTE. The protein is then refolded in a redox buffer containing oxidized glutathione and L-arginine.

One of skill would recognize that modifications can be made to the sialic acid ABC transporter protein proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

II. Preparation of Antibodies that Specifically Bind Sialic Acid ABC Transporter proteins.

Using the nucleic acid and/or protein sequences provided herein, antibodies that specifically bind one or more proteins comprising a sialic acid ABC transporter are readily prepared using any of a number of methods known to those of skill in the art. Such methods include, but are not limited to immunization methods and/or various display techniques.

A) Preparation Using Phage-Display Libraries.

In certain embodiments, antibodies that specifically bind a sialic ABC transporter polypeptide are produced in a phage display or yeast display library expressing, e.g., human scFv (see, e.g., Marks et al. (1991) J. Mol. Biol., 222: 581-597, Vaughn et al. (1996) Nature Biotechnology, 14(3): 309-314). Display of antibody fragments on the surface of viruses which infect bacteria (bacteriophage or phage) or yeast, makes it possible to produce, e.g., human antibodies (e.g., scFvs) with a wide range of affinities and kinetic characteristics. To display antibody fragments on the surface of phage (phage display), an antibody fragment gene is inserted into the gene encoding a phage surface protein (pIII) and the antibody fragment-pIII fusion protein is expressed on the phage surface (McCafferty et al. (1990) Nature, 348: 552-554; Hoogenboom et al. (1991) Nucleic Acids Res., 19: 4133-4137). For example, a scFv gene coding for the V_{H} and V_{L} domains of an anti-lysozyme antibody (Dl.3) was inserted into the phage gene III resulting in the production of phage with the DI.3 scFv joined to the N-terminus of pIII thereby producing a “fusion” phage capable of binding lysozyme (McCafferty et al. (1990) Nature, 348: 552-554,).

Since the antibody fragments on the surface of the phage are functional, phage bearing antigen binding antibody fragments can be separated from non-binding or lower affinity phage by antigen affinity chromatography (McCafferty et al. (1990) Nature, 348: 552-554). Mixtures of phage are allowed to bind to the affinity matrix bearing the antigen of interest (e.g., a SatA, SatB, SatC, SatD protein or subsequence thereof).), non-binding or lower affinity phage are removed by washing, and bound phage are eluted by treatment with acid or alkali. Depending on the affinity of the antibody fragment, enrichment factors of 20 fold-1,000,000 fold are obtained by single round of affinity selection. By infecting bacteria with the eluted phage, however, more phage can be grown and subjected to another round of selection. In this way, an enrichment of 1000 fold in one round becomes 1,000,000 fold in two rounds of selection (McCafferty et al. (1990) Nature, 348: 552-554). Thus, even when enrichments in each round are low (Marks et al. (1991) J. Mol. Biol., 222: 581-597), multiple rounds of affinity selection leads to the isolation of rare phage and the genetic material contained within which encodes the sequence of the binding antibody. The physical link between genotype and phenotype provided by phage display makes it possible to test every member of an antibody fragment library for binding to antigen, even with libraries as large as 100,000,000 clones. For example, after multiple rounds of selection on antigen, a binding scFv that occurred with a frequency of only 1/30,000,000 clones was recovered (Marks et al. (1991) J. Mol. Biol., 222: 581-597). In a particularly preferred embodiment, anti-HIV coreceptor antibodies (e.g., anti-CCR5 scFv, anti-CXCR4 scFv, anti-CCR3 scFv) are produced according to the methods of Marks et al. (1991) J. Mol. Biol., 222: 581-597), Vaughn et al. (1996) Nature Biotechnology, 14(3): 309-314).

Analysis of binding can be simplified by including an amber codon between the antibody fragment gene and gene III. This makes it possible to easily switch between displayed and soluble antibody fragments simply by changing the host bacterial strain. When phage are grown in a supE suppressor strain of E. coli, the amber stop codon between the antibody gene and gene III is read as glutamine and the antibody fragment is displayed on the surface of the phage. When eluted phage are used to infect a non-suppressor strain, the amber codon is read as a stop codon and soluble antibody is secreted from the bacteria into the periplasm and culture media (Hoogenboom et al. (1991) Nucleic Acids Res., 19: 4133-4137). Binding of soluble scFv to antigen can be detected, e.g., by ELISA using a murine IgG monoclonal antibody (e.g., 9E10) which recognizes a C-terminal myc peptide tag on the scFv (Evan et al. (1985) Mol. Cell Biol., 5: 3610-3616; Munro et al. (1986) Cell, 46: 291-300), e.g., followed by incubation with polyclonal anti-mouse Fc conjugated to horseradish peroxidase.

To facilitate purification, the anti-sialic acid ABC transporter scFv gene may be subcloned into the expression vector pUC119 Sfi-NotmycHIS which results in the addition of the myc peptide tag followed by a hexa-histidine tag at the C-terminal end of the scFv. The vector also encodes the pectate lyase leader sequence which directs expression of the scFv into the bacterial periplasm where the leader sequence is cleaved. This makes it possible to harvest native properly folded scFv directly from the bacterial periplasm. Native anti-HIV coreceptor scFv may be expressed and purified from the bacterial supernatant using immobilized metal affinity chromatography. The expression and purification of anti-HIV coreceptor(anti CCR5) scFvs is illustrated below in Example 1.

B) Antibody Preparation by Immunization.

The sialic acid ABC transporter polypeptides described herein, or fragments thereof, can be used to produce antibodies specifically reactive with sialic acid ABC transporter. In certain embodiments recombinant or synthetic polypeptides of 10 amino acids in length, or greater, selected from amino acid sub-sequences of SEQ ID NOs 5, 6, 7, or 8 can be suitable polypeptide immunogens (antigen) for the production of monoclonal or polyclonal antibodies. In one class of embodiments, an immunogenic peptide conjugate is also included as an immunogen. Naturally occurring polypeptides are also used either in pure or impure form.

The polypeptides may be chemically synthesized (as described above), purified from natural sources, or recombinantly expressed, e.g., in eukaryotic or prokaryotic cells (as described below) and purified using standard techniques. The polypeptide, or a synthetic version thereof, is then injected into an animal capable of producing antibodies.

Methods of producing polyclonal antibodies are known to those of skill in the art. In brief, an immunogen (antigen), preferably a purified polypeptide, a polypeptide coupled to an appropriate carrier (e.g., GST, keyhole limpet hemanocyanin, etc.), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired (see, e.g., Coligan (1991) Current Protocols in Immunology Wiley/Greene, N.Y.; and Harlow and Lane (1989) Antibodies: A Laboratory Manual Cold Spring Harbor Press, NY).

Antibodies, including binding fragments and single chain recombinant versions thereof, against the polypeptide sequence(s) provided herein can be raised by immunizing animals, e.g., with conjugates of the fragments with carrier proteins as described above. Typically, the immunogen of interest is a peptide of at least about 5 amino acids, more typically the peptide is 10 amino acids in length, preferably, the fragment is 15 amino acids in length and more preferably the fragment is 20 amino acids in length or greater. The peptides are typically coupled to a carrier protein (e.g., as a fusion protein), or are recombinantly expressed in an immunization vector. Antigenic determinants on peptides to which antibodies bind are typically 3 to 10 amino acids in length.

Monoclonal antibodies are prepared from cells secreting the desired antibody. These antibodies are screened for binding to the target polypeptide Specific monoclonal and polyclonal antibodies will usually bind with a K_{D} of at least about 0.1 mM, more usually at least about 50 μM, and most preferably at least about 1 μM or better.

In some instances, it is desirable to prepare monoclonal antibodies from various mammalian hosts, such as mice, rodents, primates, humans, etc. Description of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, Supra; Goding (1986) Monoclonal Antibodies: Principles and Practice (2d ed.) Academic Press, New York, N.Y.; and Kohler and Milstein (1975) Nature 256: 495-497. Summarized briefly, this method proceeds by injecting an animal with an immunogen. The animal is then sacrificed and cells taken from its spleen, which are fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein Barr Virus, oncogenes, or retroviruses, or other methods known in the art. Colonies arising from single immortalized cells are screened for production of antibodies of the desired specificity and affinity for the antigen, and yield of the monoclonal antibodies produced by such cells is enhanced by various techniques, including injection into the peritoneal cavity of a vertebrate (preferably mammalian) host. The polypeptides and antibodies of the present invention are used with or without modification, and include human as well as chimeric antibodies such as humanized murine antibodies.

III. Inhibition of Growth, and/or Proliferation, and/or Infectivity of Bacteria.

In certain embodiments this invention provides methods of inhibiting the growth and/or proliferation and/or infectivity of various bacteria, particularly bacteria expressing a sialic acid ABC transporter. The methods typically involve administering to an organism in need thereof an agent that binds to or otherwise inhibits expression and/or activity of a sialic acid ABC transporter in an amount sufficient to reduce growth and/or proliferation and/or infectivity of the bacteria.

In various embodiments the agents include, but are not limited to sialic acid analogues, antibodies that specifically bind the receptor, RNAi constructs that inhibit expression of the transporter component(s), antigen that induce a host immune response directed against the bacteria, and the like.

In certain embodiments the inhibitory agent is a viral neuraminidase inhibitor (e.g., 3-fluoro-N-acetylneuarmainic acid (3FNA), N-acetyl-2,3-didehydro-2-deoxyneuraminic acid (DDNA), the 4-guanidino-derivative of DDNA (Relenza®), Tamiflu®, and the like). In certain embodiments the inhibitor is a modified sialic acid (e.g., an N-alkanoyl-derivative of sialic acid such as 5-N-octanoyl derivative of sialic acid (SiaOct), and the like).

IV. Screening for Agents that Inhibition of Growth, and/or Proliferation, and/or Infectivity of Bacteria.

As indicated above, in one aspect, this invention pertains to the of a novel sialic acid ABC transporter that provides a good target for screening for therapeutics that inhibit the growth and/or proliferation and/or infectivity of various bacteria. Agents that inhibit expression or activity of the transporter or components thereof are expected to have therapeutic utility as described herein. Thus, in certain embodiments this invention provides methods of screening for agents that inhibit the activity and/or expression of one or more components of the sialic acid ABC transporter.

The methods typically involve detecting alteration(s) of expression and/or activity of the protein(s) of interest (e.g., SatA, SatB, SatC, SatD, etc.), and/or of the entire transporter in response to administration of one or more test agent(s) to a cell, tissue, or animal. In certain embodiments, decreased expression level, or activity level, resulting from treatment by the agent as compared to a negative control where the test agent is absent or at reduced concentration indicates that the agent down-regulates expression or activity of the factor(s).

A) Assaying for Modulators of Transporter Protein Expression.

Expression levels of a gene can be altered by changes in by changes in the transcription of the gene product (i.e., transcription of mRNA), and/or by changes in translation of the gene product (i.e., translation of the protein), and/or by post-translational modification(s) (e.g. protein folding, glycosylation, etc.). Thus preferred assays of this invention typically contacting a test cell, tissue, or animal with one or more test agents, and assaying for level of transcribed mRNA (or other nucleic acids derived from the neurotrophic and/or neurogenerative factor gene(s)), level of translated protein, activity of translated protein, etc. Examples of such approaches are described below.

1) Nucleic-Acid Based Assays.

a. Target Molecules.

Changes in expression level can be detected by measuring changes in mRNA and/or a nucleic acid derived from the mRNA (e.g. reverse-transcribed cDNA, etc.). In order to measure transporter protein expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of factor(s) of interest in a sample, the nucleic acid sample is one in which the concentration of the transporter component or the concentration of the nucleic acids derived from the ABC transporter gene(s) and/or mRNA transcript(s) encoding one or more of these components, is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript, or large differences or changes in nucleic acid concentration are desired, no elaborate control or calibration is required.

In the simplest embodiment, the nucleic acid sample is the total mRNA or a total cDNA isolated and/or otherwise derived from a biological sample (e.g., a sample from a neural cell or tissue). The nucleic acid may be isolated from the sample according to any of a number of methods well known to those of skill in the art as indicated above.

b. Hybridization-Based Assays.

Using the known sequence of sialic acid ABC transporter components, detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed transporter mRNA), typically fragmented and separated on an electrophoretic gel, is hybridized to a probe specific for the nucleic acid encoding the transporter protein(s). Comparison of the intensity of the hybridization signal from the target specific probe with a “control” probe (e.g. a probe for a “housekeeping gene) provides an estimate of the relative expression level of the target nucleic acid.

Alternatively, the sialic acid ABC transporter RNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes can be used to identify and/or quantify the target mRNA. Appropriate controls (e.g. probes to housekeeping genes) can provide a reference for evaluating relative expression level.

An alternative means for determining the transporter component expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use can vary depending on the particular application.

In some applications it is necessary to block the hybridization capacity of repetitive sequences. Thus, in some embodiments, tRNA, human genomic DNA, or Cot-1 DNA is used to block non-specific hybridization.

c. Amplification-Based Assays.

In another embodiment, amplification-based assays can be used to measure transporter component expression (transcription) level. In such amplification-based assays, the target nucleic acid sequences (e.g., transporter nucleic acid(s)) act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. healthy tissue or cells unexposed to the test agent) controls provides a measure of the transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One suitable internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The nucleic acid sequence(s) provided herein are sufficient to enable one of skill to routinely select primers to amplify any portion of the gene(s).

d. Hybridization Formats and Optimization of hybridization

i. Array-Based Hybridization Formats.

In certain embodiments, the methods of this invention can be utilized in array-based hybridization formats. Arrays typically comprise a multiplicity of different “probe” or “target” nucleic acids (or other compounds) attached to one or more surfaces (e.g., solid, membrane, or gel). In certain embodiments, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

ii. Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

e. Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results, and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

f. Labeling and Detection of Nucleic Acids.

The probes used herein for detection of sialic acid ABC transporter component expression levels can be full length or less than the full length of the mRNA(s) encoding the particular component(s) of interest. Shorter probes are empirically tested for specificity. Preferred probes are sufficiently long so as to specifically hybridize with the target nucleic acid(s) under stringent conditions. The preferred size range is from about 20 bases to the full length of the encoding mRNA, more preferably from about 30 bases to the length of the mRNA, and most preferably from about 40 bases to the length of mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

2) Polypeptide-Based Assays.

The sialic acid ABC transporter polypeptide(s) (e.g., SatA, SatB, SatC, SatD, etc.) can be detected and quantified by any of a number of methods well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the sialic acid ABC transporter polypeptide(s) are detected/quantified in an electrophoretic protein separation (e.g., a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and can be directly labeled or alternatively may be subsequently detected using labeled antibodies (e.g., labeled sheep anti-mouse antibodies) that specifically bind to the a domain of the antibody.

In certain embodiments, the sialic acid ABC transporter polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (e.g., sialic acid ABC transporter proteins). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (e.g. SatA protein) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind, e.g., sialic acid ABC transporter polypeptide(s), either alone or in combination. In the case where the antibody that binds the polypeptide(s) is not labeled, a different detectable marker, for example, an enzyme-labeled antibody capable of binding to the monoclonal antibody which binds the transporter polypeptide, can be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the sialic acid ABC transporter polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced using standard methods well know to those of skill in the art.

It will also be recognized that antibodies can be prepared by any of a number of commercial services (e.g., Berkeley antibody laboratories, Bethyl Laboratories, Anawa, Eurogenetec, etc.).

3) Reporter Based Assays.

In certain embodiments, the assays described herein utilize reporters to evaluate changes in sialic acid ABC transporter activity. The reporters typically comprise reporter genes operably linked to promoters regulating transcription of one or more component in transporter (e.g., SatA, SatB, SatC, SatD). When the pathway is upregulated, the reporter gene expression is thereby increased and readily detected. Suitable reporter genes include, but are not limited to chloramphenicol acetyl transferase (CAT), luciferase, β-galactosidase (β-gal), alkaline phosphatase, horse radish peroxidase (HRP), and green fluorescent protein (GFP), red fluorescent protein (RFP), and the like.

4) Assays for Component Activity.

In certain embodiments, the test agent(s) can be evaluated for their ability to alter (e.g., increase) the activity of the sialic acid ABC transporter. Such activity of a test agent might be via agonistic activity, e.g., at the sialic acid binding site. Thus for example, the test agent can be evaluated for its ability to inhibit sialic acid binding by the transporter and/or sialic acid uptake.

5) Assay Optimization.

The assays of this invention have immediate utility in screening for agents that inhibit the expression or activity a sialic acid ABC transporter by a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription of the sialic acid ABC transporter gene(s), nucleic acid based assays are preferred.

Routine selection and optimization of assay formats is well known to those of ordinary skill in the art.

V. Pre-Screening for Agents that Bind One or More Components of the Sialic Acid ABC Transporter.

In certain embodiments it is desired to pre-screen test agents for the ability to interact with (e.g. specifically bind to) a sialic acid ABC transporter or a component thereof, or to a nucleic acid encoding a component of the sialic acid ABC transporter. Specifically binding test agents are more likely to interact with the sialic acid ABC transporter and thereby inhibit activity or expression of the transporter. Thus, in some preferred embodiments, the test agent(s) are pre-screened for binding to a sialic acid ABC transporter component or a nucleic acid encoding such, instead of or before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In certain binding assays, the sialic acid ABC transporter or the nucleic acid encoding the sialic acid ABC transporter component is immobilized and exposed to a test agent (which can be labeled), or alternatively, the test agent(s) are immobilized and exposed to sialic acid ABC transporter component(s) or to a nucleic acid encoding such. The immobilized moiety is then washed to remove any unbound material and the bound test agent or bound protein or nucleic acid is detected (e.g. by detection of a label attached to the bound molecule). The amount of immobilized label is proportional to the degree of binding between the test agent and the sialic acid ABC transporter component(s) or to a nucleic acid encoding such.

### VI. Scoring the Assays.

As indicated above, methods of screening for modulators of expression or activity of component(s) of the sialic acid ABC transporter typically involve contacting a cell, tissue, organism, or animal with one or more test agents and evaluating changes in nucleic acid transcription and/or translation or protein expression or activity. To screen for potential modulators, the assays described above are performed in the after administering and/or in the presence of one or more test agents using biological samples from cells and/or tissues and/or organs and/or organisms exposed to one or more test agents. The activity and/or expression level and/or interaction or the protein(s) of interest is determined and, in a preferred embodiment, compared to the activity level(s) observed in “control” assays (e.g., the same assays lacking the test agent). A difference between the nucleic acid or protein expression and/or activity and/or interaction in the “test” assay as compared to the control assay indicates that the test agent is a “modulator” of expression and/or activity and/or interaction of the desired protein(s).

In a preferred embodiment, the assays of this invention level are deemed to show a positive result, e.g. decreased expression and/or activity and/or specific binding, etc. when the measured protein or nucleic acid level or protein activity is less than the level measured or known for a control sample (e.g. either a level known or measured for a normal healthy cell, tissue or organism mammal of the same species not exposed to the or putative modulator (test agent), or a “baseline/reference” level determined at a different tissue and/or a different time for the same individual). In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

### VII. High Throughput Screening.

The assays of this invention are also amenable to “high-throughput” modalities. Conventionally, new chemical entities with useful properties (e.g., inhibition of expression and/or activity of a sialic acid ABC transporter) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, Jan 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. Nos. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, Ru, 3D Pharmaceuticals, Exton, Pa., Martek Biosciences, Columbia, Md., etc.).

B) High Throughput Assays of Chemical Libraries.

Any of the assays for agents that modulate expression and/or activity of component(s) of the sialic acid ABC transporter are amenable to high throughput screening. As described above, having determined that these components/pathways are associated with the molecular mechanisms underlying addiction, it is believe that modulators can have significant therapeutic value. Certain preferred assays detect increases of transcription (i.e., increases of mRNA production) by the test compound(s), increases of protein expression by the test compound(s), or binding to the gene (e.g., gDNA, or cDNA) or gene product (e.g., mRNA or expressed protein) by the test compound(s).

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### VII. Kits.

In still another embodiment, this invention provides kits for practice of the assays or use of the compositions described herein. In one preferred embodiment, the kits comprise one or more containers containing antibodies and/or nucleic acid probes and/or substrates suitable for detection of components of the sialic acid ABC transporter and/or activity levels. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In another embodiment, the kits can comprise a container containing a sialic acid ABC transporter protein, and/or a vector encoding such a protein, and/or a cell comprising such a vector.

In certain embodiments, the kits comprise one or more agents (e.g., tat-RACK1) that decrease expression or activity of a sialic acid ABC transporter.

In addition, the kits can optionally include instructional materials containing directions (i.e., protocols) for the practice of the therapeutic and/or assay methods of this invention or the administration of the compositions described here along with counterindications. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Identification of a Novel Sialic Acid Transporter in Haemophilus ducreyi

Haemophilus ducreyi, the causative agent of chancroid, produces a 3 lipooligosaccharide (LOS) which terminates in N-acetyllactosamine. This glycoform can be further extended by the addition of a single sialic acid residue to the terminal galactose moiety. H. ducreyi does not synthesize sialic acid, which must be acquired from the host during infection or from the culture medium when the bacteria are grown in vitro. 7 However, H. ducreyi does not have genes that are highly homologous to the genes encoding known bacterial sialic acid transporters. In this study, we identified the sialic acid transporter by screening strains in a library of random transposon mutants for those mutants that were unable to add sialic acid to N-acetyllactosamine-containing LOS.

Mutants that reacted with the monoclonal antibody 3F 11, which recognizes the terminal lactosamine structure, and lacked reactivity with the lectin Maackia amurensis agglutinin (MAA), which recognizes à-2,3-linked sialic acid, were further characterized to demonstrate that they produced a N-acetyllactosamine-containing LOS by silver stained SDS-PAGE and mass spectrometric analyses. The genes interrupted in these mutants were mapped to a four gene cluster with similarity to genes encoding bacterial ABC transporters. Uptake assays using radiolabeled sialic acid confirmed that the mutants were unable to transport sialic acid. This study is the first report of bacteria using an ABC transporter for sialic acid uptake.

### Materials and Methods

Strains and Culture Conditions.

All strains used in this study are listed in Table 1. H. ducreyi strains were cultured on chocolate agar plates at 35° C. in a 5% CO₂ atmosphere as previously described (42). Chocolate agar was supplemented with 1 mM NeuAc (Calbiochem, La Jolla, Calif.) or kanamycin at 20 μg/ml when appropriate. E. coli strains were grown on Luria-Bertani (LB) plates or in LB broth, and were supplemented with kanamycin when appropriate.

TABLE 1
Strains used in this study

| | | Source or |
|---|---|---|
| Strain | Description | reference |
| H. ducreyi | Human passaged virulent isolate | (53) |
| 3 5000HP | 1st mutant | (6) |
| 3 5000HP-RSM203 | satA::mTn5 | This study |
| 35000HP-3 10 | satA::mTn5 | This study |
| 35000HP-320 | satA::mTn5 | This study |
| 35000HP-3 19 | satB::mTn5 | This study |
| 35000HP-305 | satB::mTn5 | This study |
| 35000HP-306 | satC::mTn5 | This study |
| 35000HP-322 | satD::mTn5 | This study |

Construction and Screening of a Transposome Library.

Several colonies from a 48 h 10 chocolate agar plate of strain 35000HP were swabbed with a cotton swab onto a fresh chocolate agar plate. After incubation for 12 to 14 h at 35° C. in a 5% CO₂ atmosphere, competent cells were prepared as described by Palmer and co-workers (42). Electrocompetent cells were electroporated with 1 μl of EZ::TN<R6Käori/KAN-2> transposome (Epicentre, Madison, Wis.) then suspended in 100 μl of brain heart infusion broth and spread on a chocolate agar plate. After 6 h of incubation, cells were scraped and dilutions were plated on chocolate agar containing kanamycin at 20 μg/ml. After 48 h, approximately 2500 clones were patched onto chocolate agar plates supplemented with kanamycin. After incubation overnight, cells were lifted onto nitrocellulose then lysed by incubation in chloroform vapor for 5 min. Non-specific protein binding sites were blocked by incubation of the filters in 1% skim milk in TBS (0.05 M Tris, 0.15 M NaCl, pH 7.5). The filter was then incubated with a 1:200 dilution of Maackia amurensis agglutinin (MAA) lectin conjugated to horseradish peroxidase (EY labs, San Mateo, Calif.) in TBS for 1 h. After 3 washes with TBS, the blot was developed with Bio-Rad peroxidase detection reagents (Bio-Rad, Hercules, Calif.). Twenty-two clones that failed to react with MAA were then screened for reactivity with the murine monoclonal antibody 3F 11 (8). Colonies were lifted and lysed as above, then the filters were processed as described by Sun and co-workers (57).

LOS Isolation and Analysis.

LOS preparations for SDS-PAGE and mass spectrometric analyses were extracted from H. ducreyi cells grown for two days on chocolate agar plates. Cells were washed with PBS (pH 7.4) containing 0.5 mM MgCl₂ and 0.15 mM CaCl₂, and suspended in ddH₂O. LOS was extracted using the hot phenol method (27). One-tenth of the total LOS extracted from each sample was set aside for SDS-PAGE analysis. These samples were reconstituted in 10 μl of ddH₂O, 1-2 μl of these samples were diluted in 8-9 μl of Laemmli sample buffer containing 2% α-mercaptoethanol (BioRad), for a total volume of 10 μl. Neuraminidase treated LOS samples were reconstituted in 5 μl of 2× neuraminidase buffer (100 mM sodium acetate, 8 mM calcium chloride, pH 5.5) and incubated overnight at 37° C. with 5 mU of neuraminidase isolated from Vibrio cholerae (5 μl of a 1 U/ml solution, Roche, Indianapolis, Ind.). These samples were diluted in Laemmli buffer as described above. All SDS-PAGE samples were boiled for 10 min, followed by a brief centrifugation, and 1-2 μl of each sample were loaded onto the gel. Samples were separated on a 15% SDS-PAGE gel (32). Silver staining was performed according to a previously described protocol by Tsai and Frasch (64).

Mass Spectrometry Analysis.

Water soluble O-deacylated LOS(O-LOS) samples were prepared by treating the LOS extracted from one agar plate with 50 μl of anhydrous hydrazine followed by acetone precipitation (44). Lyophilized O-LOS samples were desalted by reconstituting in ddH2O and performing drop dialysis using 0.025 μm pore size nitrocellulose membranes (Millipore, Bedford, Mass.). Samples were then lyophilized and reconstituted in 5-10 μl of ddH2O. O-LOS samples were further de-salted using 3+ form). Before loading samples Dowex 50×100-200 mesh cation exchange beads (NH4 onto the target, they were mixed 1:1 (v/v) with matrix (160 mM 2,5-dihydroxybenzoic acid, 87.5 mM 1-hydroxyisoquinoline solution in acetone-water (4:1, v/v). For the analysis of O-LOS, matrix-assisted laser desorption ionization mass spectrometry (MALDI-MS) using an Applied Biosystems (Framingham, Mass.) Voyager DE time-of-flight mass spectrometer was performed. Mass spectra were run in linear negative-ion mode with a nitrogen laser (337 nm) under delayed extraction conditions: 165 ns delay time, with a grid voltage of 94% of full acceleration voltage (20 kV). Mass spectra were acquired, averaged (typically 100 laser shots), and externally calibrated with a standard peptide mixture consisting of angiotension I, and ACTH fragments 1-7, 18-39, and 7-38 (Bachem, Torrance, Calif.). All masses measured under these conditions were average masses.

Identification of the Genes Inactivated by mTn5 in the Clones that were MAA-Negative and 3F11-Positive.

The sequences flanking the Tn5 element were first rescued and then sequenced. Genomic DNA was prepared from each clone using the Puregene reagents (Gentra Systems, Minneapolis, Minn.), digested with MfeI, self ligated and transformed into E. coli EC100D pir⁺ (Epicentre). DNA sequence flanking the mTn5 insertion was then determined in both directions using dye terminator chemistries and oligonucleotide primers KAN-2 FP-1 and R6KAN-2 RP-1 (Epicentre). No clones were rescued from strain 35000HP-310. However, sequence flanking the mutation in this strain was determined using a single primer PCR methodology as described by Ducey and Dyer (15). The resulting sequences were used to query the H. ducreyi genome sequence using the BLAST algorithm (4) and Seqman from the DNASTAR suite of programs.

Sialic Acid Transport.

Several colonies from a 48 h chocolate agar plate of each strain were swabbed with a cotton swab onto a fresh chocolate agar plate. After incubation for 12 to 14 h at 35° C. in a 5% CO₂ atmosphere, cells were suspended in RPMI 1640 without phenol red (ICN, Aurora, Ohio) supplemented with 1% (v/v) 200 mM glutamine (Invitrogen, Carlsbad, Calif.), 1% (v/v) 100 mM sodium pyruvate (Invitrogen) and 10% (v/v) 0.5 M Hepes, pH 7.5, then harvested by centrifugation at room temperature for 10 min at 3200×g. Cells were resuspended to an absorbance of 2.0 at 600 nm in the same medium. An aliquot of the cell suspension was equilibrated to 35° C. for 3 min and the reaction started by addition of 1/10 volume of RPMI-based medium containing [³H]-sialic acid (American Radiochemicals, St. Louis, Mo.) prepared such that the concentration of sialic acid (Sigma, St. Louis, Mo.) in the final reaction mixture was 2.5 μM. After the indicated period of time, the reaction mixture was rapidly filtered through a 0.22 μm type GS filter (Millipore, Bedford, Mass.) and washed twice with 2 ml of RPMI-based medium. Filters were air dried, then counted in 10 ml of ScintiSafe 30% LSC-Cocktail (Fischer Scientific, Hampton, N.H.) in a Beckman LS 6500 Scintillation Counter (Beckman Coulter, Fullerton, Calif.).

### Results

Construction of a Library of Transposon Mutants and Identification of Strains Lacking Sialic Acid in their LOS.

Mutagenesis was performed by introducing a mTn5 transposon randomly into the chromosome of H. ducreyi. Kanamycin resistant clones were screened with the MAA lectin, which recognizes terminal α2,3-linked sialic acid. Clones that did not bind MAA were then screened with the monoclonal antibody 3F1 1, which recognizes terminal N-acetyllactosamine. Nine clones were identified that were 3F1 1 positive and MAA negative (Table 1).

SDS-PAGE Analysis of LOS.

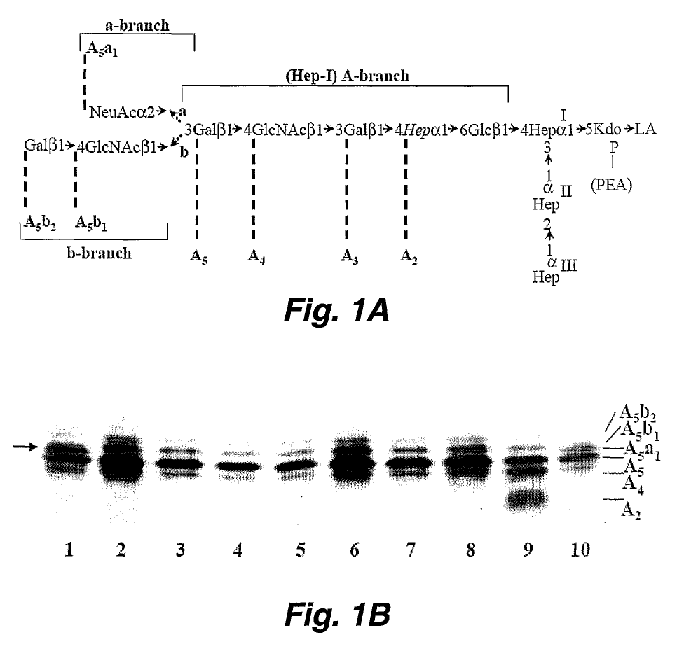

LOS from seven of the mutants, 35000HP-305, -306, -310, -313, -319, -320, and -322 was isolated and compared, by SDS-PAGE, with LOS isolated from strains 35000HP and 35000HP-RSM203, a sialyltransferase (1st) mutant (6). The structure of 35000HP LOS has been determined and nomenclature describing its various glycoforms has been developed, both are shown in FIG. 1A (6). Silver stained SDS-PAGE gels demonstrated that the previously identified sialylated glycoform (band A₅a₁)(39) was absent from LOS isolated from all seven mutants generated in this study, as well as the sialyltransferase mutant 35000HP-RSM203 (FIG. 1B). The A₅b₁ and A₅b₂ bands, corresponding to the addition of GlcNAc or Gal-β1,4-GlcNAc, respectively, were very prevalent in all of the mutants, and in the LOS from the sialyltransferase mutant, 35000HP-RSM203. FIG. 1C shows a representative SDS-PAGE of LOS isolated from the parent strain 35000HP, the 1st mutant 35000HP-RSM203, and the mutant 35000HP-310 with or without neuraminidase treatment. In the neuraminidase treated 35000HP LOS sample the sialylated band (A₅a₁) was not visible. In addition, the di-N-acetyllactosamine band (A₅b₁) was only visible in the 35000HP LOS sample after neuraminidase treatment. Neuraminidase treatment had no visible affect on either the LOS from strain 35000HP-310 or the LOS from the sialyltransferase mutant 35000HP-RSM203. LOS from all of the mutants was treated with neuraminidase prior to separation by SDS-PAGE. This treatment had no visible affect on the LOS isolated from the mutants (data not shown). The LOS from the other 2 MAA negative and 3F1 1 positive mutants did not contain the A₅b₁ and A₅b₂ LOS-glycoforms (data not shown), and therefore were not further characterized in this study.

Mass Spectrometric Analysis of O-LOS.

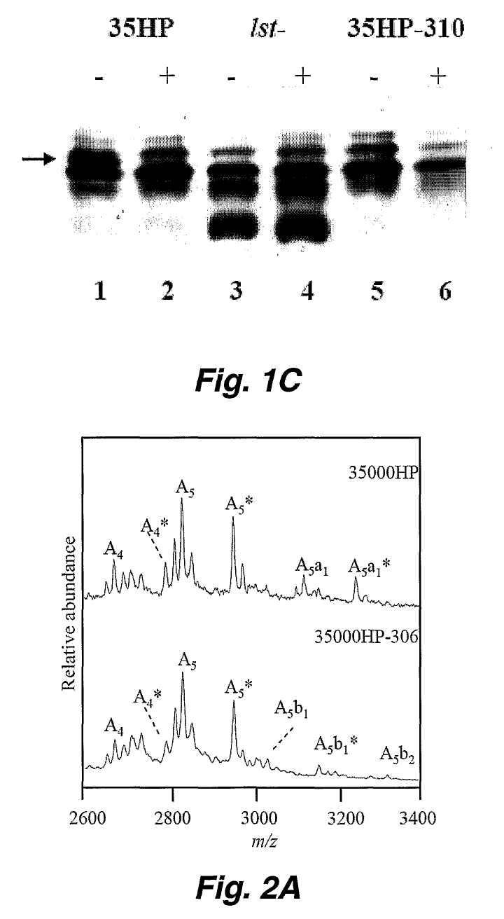

Mass spectra obtained from O-LOS isolated from the parent strain 35000HP and strain 35000HP-306 are shown in FIG. 2. For clarity, only the full O-LOS region of the spectra are shown since there were no differences observed in the lipid A region of the mutant compared to the parent strain. The LOS from 35000HP has been previously characterized, and a nomenclature describing the various LOS glycoforms was established (FIG. 1A) (6). The A₅* and A₅ peaks, corresponding to LOS that terminate in N-acetyllactosamine (with and without PEA, respectfully), are the most prevalent peaks in all of the spectra (FIG. 2A, 2B). However, the A₅a₁ and A₅a₁* peaks, previously identified as the A₅ and A₅* structures with the addition of one NeuAc, respectively, are only present in the 35000HP O-LOS. Previous experiments from our laboratory demonstrated that supplementing the growth media of H. ducreyi with additional sialic acid increases the abundance of the sialylated glycoforms present in H. ducreyi LOS (50). FIG. 2B shows the O-LOS spectra from 35000HP and 35000HP-306 grown in media supplemented with 1 mM sialic acid. The relative abundance of the A₅a₁* peak is increased in the 35000HP sample, under these conditions of high sialic acid, corresponding to the previous findings (50). However, even when grown in the presence of 1 mM sialic acid, the sialylated peaks, A₅a₁ and A₅a₁*, are not detectable in the 35000HP-306 sample. FIGS. 2A and 2B both demonstrate that the A₅b₁, A₅b₁*, and A₅b₂ peaks, corresponding to the addition of a GlcNAc (without and with PEA) and lactosamine to the A₅ structure, respectively, are more readily detectable in the 35000HP-306 samples compared to the 35000HP samples. These data correspond to previous findings (6), and indicate that the absence of the sialic acid on the terminal Gal of the A₅ LOS-glycoform enables a larger percent of the LOS to extend the oligosaccharide to a di-N-acetyllactosamine. These data are representative of the seven mutants characterized in this study.

Identification of the Genes Insertionally Inactivated by mTn5 in the Mutants.

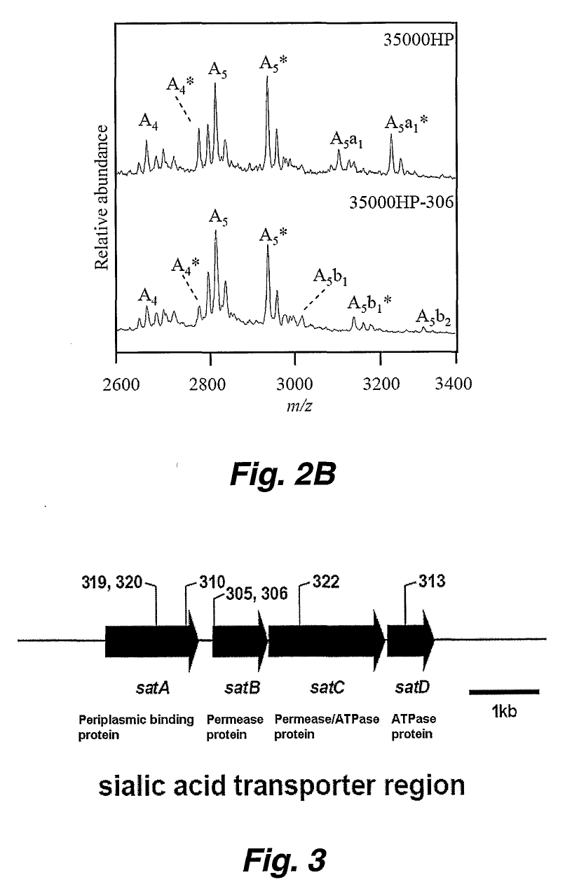

Sequences flanking the transposon insertion in the seven mutants were sequenced. Surprisingly, none of these insertions mapped to either the sialyltransferase gene, 1st, or the CMP-NeuAc synthetase, neuA. Instead, these mutants all had Tn5 insertions in the genes designated Hd1669-1672 (Table 1, FIG. 3). This gene cluster had previously been predicted to encode an ABC-transporter. Based on sequence analysis and phenotype, the Hd1669-1672 genes were designated satABCD (sat for sialic acid transport), respectively. The Hd1669 gene (satA) has 2 possible AUG start codons. When translated from the second AUG, the gene is predicted to encode for a protein with a Mᵣ of 56,495. As translated, lysine is present at amino acid position 2 and ADA residues are present at positions 22-24. AXA is frequently present at a signal peptidase cleavage site. Together with the lysine at position 2, this sequence is typical of leader peptides suggesting that Hd1669 contains a 24 amino acid leader peptide. Blast analysis indicates that this protein is a member of the family 5 of the bacterial extracellular solute-binding proteins (pfam00496). Family 5 includes the oligopeptide and dipeptide binding proteins OppA and DppA of E. coli, a number of other gram-negative periplasmic binding proteins, and gram-positive homologues of these periplasmic proteins which are lipoproteins. The Hd1670 and Hd1671 genes (satB and satC, respectively) are predicted to encode for the integral membrane permease proteins with a Mr of 35,134 and a Mᵣ of 71,259, respectively. Hd1670 is a member of pfam00528, a family of binding protein-dependent transport system integral inner membrane proteins. Hd1671 has two domains; the N-terminal domain that shows high homology to other permease proteins and the C-terminal domain that has high homology to transport ATPases (NCBI conserved domain cd0267.1). The Hd1672 gene (satD) is predicted to encode for a protein with a Mr of 29,593. This protein also contains a transport ATPase domain of the same class (NCBI conserved domain cd0267.1). The predicted protein sequence from SatD matched the consensus sequence of the critical conserved domains of ABC ATPases, the sequence from SatC matched the consensus sequences except for the addition of one amino acid in the Walker A motif.

Sialic Acid Uptake Assay.

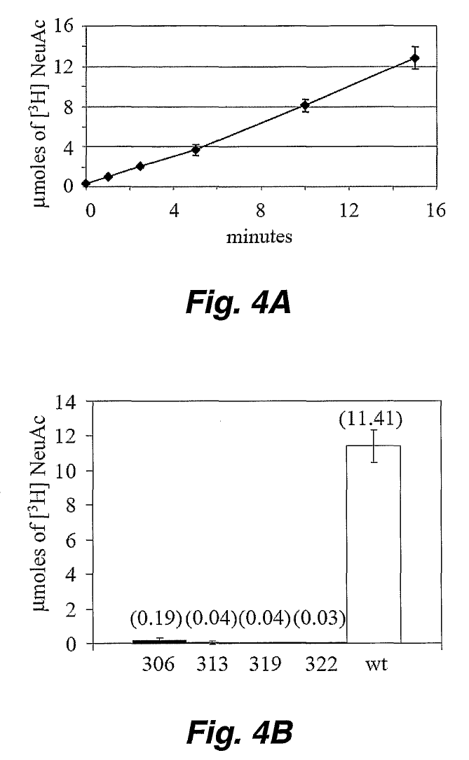

ABC transport systems with periplasmic binding proteins are considered to be uptake systems. This observation together with the lack of sialic acid in the LOS of the mutants is consistent with the hypothesis that Hd1669-1672 encodes the sialic acid transporter. However, none of the evidence presented unequivocally demonstrates that this gene cluster encodes the sialic acid transporter. Therefore, the mutants were directly tested for their ability to transport sialic acid. First, sialic acid transport by the parental strain was demonstrated by incubation of the parental strain with [³H]-NeuAc followed by filtration to remove sialic acid that was not cell-associated. FIG. 4A shows a time course experiment. These data show that the bacteria are able to transport [³H]-NeuAc under these experimental conditions. Representative ABC transporter mutants were also tested. FIG. 4B shows single 10 min time-point data for cells of strains 35000HP-306 (satB mutant), 35000HP-313 (satD mutant), 35000HP-319 (satA mutant), 35000HP-322 (satC mutant), and 35000HP. These data demonstrated that all four mutants are deficient in the transport of [³H]-NeuAc compared to the parental strain, 35000HR.

### Discussion

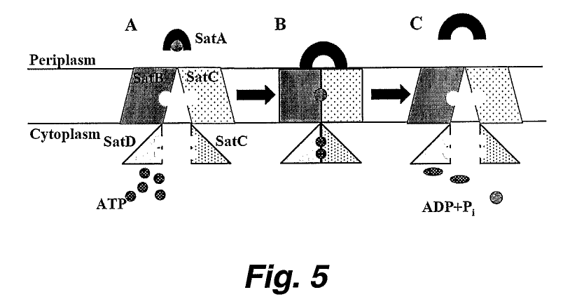

In this study, we have shown that H. ducreyi utilizes the products of the satABCD genes to encode an ABC transporter for sialic acid uptake. Sequence comparisons with other ABC transporter systems suggest that these genes encode the periplasmic binding protein (PBP), SatA, the integral-membrane permease protein, SatB, and the ATPase, SatD. These comparisons also show that the N-terminus of the SatC protein contains a permease domain while the C-terminus contains an ATPase domain. How might sialic acid be transported by this novel ABC transporter? A hypothetical model for sialic acid transport in H. ducreyi, based on the predicted protein functions, can be constructed as shown in FIG. 5. This model is based on the maltose transporter in E. coli (12). The sialic acid transporter model predicts that sialic acid is first bound to the PBP, SatA. Binding of the sialic acid may increase the affinity of SatA for SatB/SatC. Binding of the SatA-sialic acid complex to SatB/SatC may decrease SatA's affinity for sialic acid. This decrease may allow for the release of sialic acid, the reorientation of SatB/SatC, and the exposure of sites needed for ATP hydrolysis. After ATP hydrolysis, sialic acid is transported to the cytoplasm and the rest of the complex returns to its original conformation.

The sialic acid transporter identified in H. ducreyi is the first ABC transporter identified that transports sialic acid; however, sequence analysis suggests that it may be the first of many bacterial sialic acid ABC transporters yet to be identified and characterized (67). In addition to H. ducreyi, two other members of the Pasteurellaceae family, Actinobacillus pleuropneumoniae and Haemophilus somnus, may utilize an ABC transporter for sialic acid uptake. Both bacteria have proteins which are highly homologous to the H. ducreyi SatABCD proteins. Proteins predicted from unfinished genome sequences of A. pleuropneumoniae serovar 1 strain 4074 and H. somnus 1 29PT are 86% identical (over 510 amino acids) and 75% identical (over 454 amino acids) to SatA from H. ducreyi, respectively. The H. ducreyi SatB protein is 92% identical (over 317 amino acids) to a predicted protein from A. pleuropneumoniae serovar 1 strain 4074, and 84% identical (over 317 amino acids) to predicted proteins from H. somnus 1 29PT and H. somnus 2336. The H. ducreyi SatC protein is 85% identical (over 648 amino acids) to a predicted protein from A. pleuropneumoniae serovar 1 strain 4074, 81% identical (over 649 amino acids) to a predicted protein from H. somnus 2036 and 80% identical (over 649 amino acids) to a predicted protein from H. somnus 2336. Similarly, the SatD protein is highly homologous to predicted proteins from A. pleuropneumoniae and H. somnus. These data strongly suggest that sialic acid is also transported by an ABC transporter in these members of the Pasteurellaceae.

The gram-positive bacteria Corynebacterium glutamicum, Corynebacterium diphtheriae, and Streptomyces avermitilis, also have homologues of the sat genes. The satA gene of H. ducreyi has a 40% G+C content, similar to the overall G+C content of the H. ducreyi genome (3 8.2% G+C). In contrast, these gram-positive organisms have a high G+C content both in their respective genomes and in their satA homologues, suggesting that the sat genes were not recently passed to or acquired from each other.

Mechanisms of sialic acid transport are diverse among bacteria, even within their subfamilies. The sialic transporter in E. coli, NanT, is a symporter of the major facilitator superfamily type of membrane transporters (38, 67). In this type of transport protons or metal ions are coupled to solute uptake (67). The tripartite ATP-independent periplasmic (TRAP) type of transporter has been proposed for sialic acid uptake in two members of the Pasteurellaceae family, Pasturella inultocida subsp. multocida and H. influenzae (55, 67). The TRAP transporters are predicted to have a periplasmic binding component, and one membrane spanning protein (55, 57). The identification that H. ducreyi, also a member of the Pasteurellaceae family, transports sialic acid using an ABC transporter adds a third method of sialic transport. Thus, there are at least three different methods of sialic acid transport in bacteria. The finding that differences in the types of transport mechanisms used by these bacteria occurs even within a family is intriguing and could represent the various needs and environments that these bacteria encounter, or alternatively, could be an example of convergent evolution.

Besides incorporation of sialic acid into LOS/lipopolysaccharide (LPS) and capsule, some bacteria are also able to utilize sialic acid as a carbon and nitrogen source (45, 68). Studies in E. coli have demonstrated that a number of genes are involved in the conversion of sialic acid to fructose-6-phosphate (45, 68). After transport, the first step in the sialic acid catabolic pathway is its conversion to N-acetylmannosamine (ManNAc) and pyruvate by the aldolase NanA (45, 68). ManNAc is then phosphorylated by the ATP-dependent kinase NanK (48), and the product, ManNAc-6-P, is then converted to N-acetylglucosamine-6-phosphate (GlcNAc-6-P) by the epimerase NanE (48). Conversion of GlcNAc-6-P to glucosamine-6-phosphate (Glc-6-P) is performed by the deacetylase NagA (45). Lastly, NagB is involved in the conversion of Glc-6-P to fructose-6-phosphate (Fru-6-P)(45). Comparisons of the E. coli genes involved in the sialic acid catabolism pathway to the H. influenzae genome demonstrated that a number of potentially homologous genes were present (66). These genes include nanA, nagA, nagB, nanK, and nanE homologues. Mutagenesis performed on the putative H. influenzae aldolase encoding gene, nanA, demonstrated that this mutant was unable to metabolize sialic acid and exhibited no detectable aldolase activity (66). Whole-cell ELISA of H. influenzae nanA mutants, with the monoclonal antibody 3F11, demonstrated that the mutants had a dramatic increase in the level of sialylation of the LOS (66). These data suggest that in these organisms there exists a balance between incorporation of sialic acid into LOS and the use of sialic acid as a carbon source. Sequence analyses of H. ducreyi strain 35000HP, with the H. influenzae sialic acid catabolic pathway genes, indicate that a region 5′ of rfe contains putative nanA, nanE, nagA and nagB homologues, but does not seem to contain a nanK homologue. Due to the apparent absence of a homologous nanK in H. ducreyi it is currently unclear if H. ducreyi possesses a functional sialic acid degradation pathway. Studies to discern whether this catabolic pathway is functional in H. ducreyi are currently underway.

Since the accumulation of high levels of sialic acid in the cytoplasm have been shown to be toxic in some bacteria (68), how might H. ducreyi control uptake and intracellular levels of sialic acid? In this and previous studies, H. ducreyi were grown on plates containing high levels of sialic acid (22, 50). The presence of the sialic acid did not seem to affect the growth of the bacteria. To more quantitatively test whether the presence of high levels of sialic acid affected the growth rate of H. ducreyi, growth curves of H. ducreyi in low (˜0.5 μM, basal level in media) (50), medium (10 μM), and high (1 mM) sialic acid conditions were performed (46). There were no differences in the growth rates suggesting that H. ducreyi either regulates sialic acid uptake, degrades sialic acid and uses it as a carbon source, or that the bacteria are able to tolerate some intracellular accumulation of sialic acid. Both E. coli and H. influenzae seem to utilize their ability to degrade sialic acid as one mechanism of controlling the intracellular levels of sialic acid (66, 68, 69). As noted above, it is currently unclear if H. ducreyi is able to degrade sialic acid. Another possible mechanism for controlling sialic acid uptake may be through the PBPs. The PBPs from other bacteria have been shown to be important for controlling the amount and efficiency of solute uptake (12, 13, 30, 31, 37, 45). Therefore, it is possible that SatA may play an important role in controlling sialic acid uptake in H. ducreyi. One mechanism of control could be that the level of satA expression may be affected by the intracellular levels of sialic acid. When the intracellular pool of sialic acid is high, expression of satA is downregulated; conversely, when the intracellular pool of sialic acid is low, expression of satA is upregulated. This type of regulation has been shown to be effective for controlling intracellular pools of iron in a wide range of bacteria (reviewed in (18)). Another possible option for H. ducreyi to manage possible toxic levels of intracellular sialic acid is to have a relatively high tolerance for the accumulation of sialic acid. Under these conditions the levels of SatA would not dramatically change under low or high sialic acid conditions, instead satA would be expressed constitutively and sialic acid uptake would not be tightly regulated. If this option is correct, it suggests that H. ducreyi have a higher tolerance, compared to E. coli, for accumulation of sialic acid in the cytoplasm. The identification of the components of the H. ducreyi sialic acid transporter should enable further investigation of the regulation of sialic acid uptake and utilization.

The role that sialylation of the LOS in H. ducreyi plays in virulence is currently unclear. However, similar to other bacteria (24, 58, 59), H. ducreyi LOS plays a role in adherence to human cells (2, 20). The exact role that LOS plays in facilitating this interaction has not yet been elucidated. Recent studies in H. influenzae demonstrated that sialylation of the LOS played a role in biofilm formation (23, 60). In addition, one of these studies demonstrated that these bacteria produce a biofilm containing á2,6-linked sialic acid (23). Sialylation of LOS in H. ducreyi could therefore play a role in adherence or possibly microcolony formation. Now that we have identified the sialic acid uptake system in H. ducreyi as a novel ABC-transporter (SatABCD), thus completing the identification of the major components of the sialic acid biosynthesis pathway, it should be possible to design experiments that will determine the role of sialic acid in the biology of this human pathogen.

### REFERENCES

1. Ahmed, H. J., A. Frisk, J. E. Mansson, E. K. Schweda, and T. Lagergard. 1997. Structurally defined epitopes of Haemophilus ducreyi lipooligosaccharides recognized by monoclonal antibodies. Infect Immun 65:3151-8.
2. Alfa, M. J., and P. DeGagne. 1997. Attachment of Haemophilus ducreyi to human foreskin fibroblasts involves LOS and fibronectin. Microb Pathog 22:3 9-46.
3. Alfa, M. J., P. DeGagne, and P. A. Totten. 1996. Haemophilus ducreyi hemolysin acts as a contact cytotoxin and damages human foreskin fibroblasts in cell culture. Infect Immun 64:2349-52.
4. Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res 25:33 89-402.
5. Apicella, M. A., R. E. Mandrell, M. Shero, M. E. Wilson, J. M. Griffiss, G. F. Brooks, C. Lammel, J. F. Breen, and P. A. Rice. 1990. Modification by sialic acid of Neisseria gonorrhoeae lipooligosaccharide epitope expression in human urethral exudates: an immunoelectron microscopic analysis. J Infect Dis 162:506-12.
6. Bozue, J. A., M. V. Tullius, J. Wang, B. W. Gibson, and R. S. Munson, Jr. 1999. Haemophilus ducreyi produces a novel sialyltransferase. Identification of the sialyltransferase gene and construction of mutants deficient in the production of the sialic acid-containing glycoform of the lipooligosaccharide. J Biol Chem 274:4106-14.
7. Campagnari, A. A., R. Karalus, M. Apicella, W. Melaugh, A. J. Lesse, and B. W. Gibson. 1994. Use of pyocin to select a Haemophilus ducreyi variant defective in lipooligosaccharide biosynthesis. Infect Immun 62:2379-86.
8. Campagnari, A. A., S. M. Spinola, A. J. Lesse, Y. A. Kwaik, R. E. Mandrell, and M. A. Apicella. 1990. Lipooligosaccharide epitopes shared among gram-negative non-enteric mucosal pathogens. Microb Pathog 8:353-62.
9. Cole, L. E., T. H. Kawula, K. L. Toffer, and C. Elkins. 2002. The Haemophilus ducreyi serum resistance antigen DsrA confers attachment to human keratinocytes. Infect Immun 70:6158-65.
10. Cope, L. D., S. Lumbley, J. L. Latimer, J. Klesney-Tait, M. K. Stevens, L. S. Johnson, M. Purven, R. S. Munson, Jr., T. Lagergard, J. D. Radolf, and E. J. Hansen. 1997. A diffusible cytotoxin of Haemophilus ducreyi. Proc Natl Acad Sci USA 94:4056-61.
11. Cortes-Bratti, X., C. Karlsson, T. Lagergard, M. Thelestam, and T. Frisan. 2001. The Haemophilus ducreyi cytolethal distending toxin induces cell cycle arrest and apoptosis via the DNA damage checkpoint pathways. J Biol Chem 276:5296-302.
12. Davidson, A. L., and J. Chen. 2004. ATP-binding cassette transporters in bacteria. Annu Rev Biochem 73:241-68.
13. De Pina, K., V. Desjardin, M. A. Mandrand-Berthelot, G. Giordano, and L. F. Wu. 1999. Isolation and characterization of the nikR gene encoding a nickel-responsive regulator in Escherichia coli. J Bacteriol 181:670-4.
14. Deng, K., and E. J. Hansen. 2003. A CdtA-CdtC complex can block killing of HeLa cells by Haemophilus ducreyi cytolethal distending toxin. Infect Immun 71:6633-40.
15. Ducey, T. F., and D. W. Dyer. 2002. Rapid identification of EZ::TN insertion sites in the genome of Neisseria gonorrhoeae. Epicentre Forum 9:6-7.
16. Elkins, C. 1995. Identification and purification of a conserved heme-regulated hemoglobin-binding outer membrane protein from Haemophilus ducreyi. Infect Immun 63:1241-5.
17. Elkins, C., K. J. Morrow, Jr., and B. Olsen. 2000. Serum resistance in Haemophilus ducreyi requires outer membrane protein DsrA. Infect Immun 68:1608-19.
18. Escolar, L., J. Perez-Martin, and V. de Lorenzo. 1999. Opening the iron box: transcriptional metalloregulation by the Fur protein. J Bacteriol 181:6223-9.
19. Frisk, A., C. A. Ison, and T. Lagergard. 1998. GroEL heat shock protein of Haemophilus ducreyi: association with cell surface and capacity to bind to eukaryotic cells. Infect Immun 66:1252-7.
20. Gibson, B. W., A. A. Campagnari, W. Melaugh, N.J. Phillips, M. A. Apicella, S. Grass, J. Wang, K. L. Palmer, and R. S. Munson, Jr. 1997. Characterization of a transposon Tn91 6-generated mutant of Haemophilus ducreyi 35000 defective in lipooligosaccharide biosynthesis. J Bacteriol 179:5062-71.
21. Gibson, B. W., W. Melaugh, N.J. Phillips, M. A. Apicella, A. A. Campagnari, and J. M. Griffiss. 1993. Investigation of the structural heterogeneity of lipooligosaccharides from pathogenic Haemophilus and Neisseria species and of R-type lipopolysaccharides from Salmonella typhimurium by electrospray mass spectrometry. J Bacteriol 175:2702-12.
22. Goon, S., B. Schilling, M. V. Tullius, B. W. Gibson, and C. R. Bertozzi. 2003. Metabolic incorporation of unnatural sialic acids into Haemophilus ducreyi lipooligosaccharides. Proc Natl Acad Sci USA 100:3089-94.
23. Greiner, L. L., H. Watanabe, N. J. Phillips, J. Shao, A. Morgan, A. Zaleski, B. W. Gibson, and M. A. Apicella. 2004. Nontypeable Haemophilus influenzae strain 2019 produces a biofilm containing N-acetylneuraminic acid that may mimic sialylated O-linked glycans. Infect Immun 72:4249-60.
24. Harvey, H. A., M. P. Jennings, C. A. Campbell, R. Williams, and M. A. Apicella. 2001. Receptor-mediated endocytosis of Neisseria gonorrhoeae into primary human urethral epithelial cells: the role of the asialoglycoprotein receptor. Mol Microbiol 42:659-72.
25. Harvey, H. A., N. Porat, C. A. Campbell, M. Jennings, B. W. Gibson, N.J. Phillips, M. A. Apicella, and M. S. Blake. 2000. Gonococcal lipooligosaccharide is a ligand for the asialoglycoprotein receptor on human sperm. Mol Microbiol 36:1059-70.
26. Harvey, H. A., W. E. Swords, and M. A. Apicella. 2001. The mimicry of human glycolipids and glycosphingolipids by the lipooligosaccharides of pathogenic Neisseria and Haemophilus. J Autoimmun 16:257-62.
27. Inzana, T. J. 1983. Electrophoretic heterogeneity and interstrain variation of the lipopolysaccharide of Haemophilus influenzae. J Infect Dis 148:492-9.
28. Jessamine, P. G., and A. R. Ronald. 1990. Chancroid and the role of genital ulcer disease in the spread of human retroviruses. Med Clin North Am 74:1417-31.
29. Kahler, C. M., L. E. Martin, G. C. Shih, M. M. Rahman, R. W. Carlson, and D. S. Stephens. 1998. The (á2-->8)-linked polysialic acid capsule and B lipooligosaccharide structure both contribute to the ability of serogroup Neisseria meningitidis to resist the bactericidal activity of normal human serum. Infect Immun 66:5939-47.
30. Kalivoda, K. A., S. M. Steenbergen, E. R. Vimr, and J. Plumbridge. 2003. Regulation of sialic acid catabolism by the DNA binding protein NanR in Escherichia coli. J Bacteriol 185:4806-15.
31. Koman, A., S. Harayama, and G. L. Hazelbauer. 1979. Relation of chemotactic response to the amount of receptor: evidence for different efficiencies of signal transduction. J Bacteriol 138:739-47.
32. Laemmli, U. K. 1970. Cleavage of structural proteins during the assembly of the head of bacteriophage T4. Nature 227:680-5.
33. Langford, P. R., and J. S. Kroll. 1997. Distribution, cloning, characterisation and mutagenesis of sodC, the gene encoding copper/zinc superoxide dismutase, a potential determinant of virulence, in Haemophilus ducreyi. FEMS Immunol Med Microbiol 17:235-42.
34. Leduc, I., P. Richards, C. Davis, B. Schilling, and C. Elkins. 2004. A novel lectin, DltA, is required for expression of a full serum resistance phenotype in Haemophilus ducreyi. Infect Immun 72:34 1 8-28.
35. Mandrell, R. E., J. M. Griffiss, and B. A. Macher. 1988. Lipooligosaccharides (LOS) of Neisseria gonorrhoeae and Neisseria meningitidis have components that are immunochemically similar to precursors of human blood group antigens. Carbohydrate sequence specificity of the mouse monoclonal antibodies that recognize crossreacting antigens on LOS and human erythrocytes. J Exp Med 168:107-26.
36. Mandrell, R. E., R. McLaughlin, Y. Aba Kwaik, A. Lesse, R. Yamasali, B. Gibson, S. M. Spinola, and M. A. Apicella. 1992. Lipooligosaccharides (LOS) of some Haemophilus species mimic human glycosphingolipids, and some LOS are sialylated. Infect Immun 60:1322-8.
37. Manson, M. D., W. Boos, P. J. Bassford, Jr., and B. A. Rasmussen. 1985. Dependence of maltose transport and chemotaxis on the amount of maltose-binding protein. J Biol Chem 260:9727-33.
38. Martinez, J., S. Steenbergen, and E. Vimr. 1995. Derived structure of the putative sialic acid transporter from Escherichia coli predicts a novel sugar permease domain. J Bacteriol 177:6005-10.
39. Melaugh, W., A. A. Campagnari, and B. W. Gibson. 1996. The lipooligosaccharides of Haemophilus ducreyi are highly sialylated. J Bacteriol 178:564-70.
40. Melaugh, W., N.J. Phillips, A. A. Campagnari, M. V. Tullius, and B. W. Gibson. 1994. Structure of the major oligosaccharide from the lipooligosaccharide of Haemophilus ducreyi strain 35000 and evidence for additional glycoforms. Biochemistry 33:13070-8.
41. Pacello, F., P. R. Langford, J. S. Kroll, C. Indiani, G. Smulevich, A. Desideri, G. Rotilio, and A. Battistoni. 2001. A novel heme protein, the Cu,Zn-superoxide dismutase from Haemophilus ducreyi. J Biol Chem 276:30326-34.
42. Palmer, K. L., W. E. Goldman, and R. S. Munson, Jr. 1996. An isogenic haemolysin-deficient mutant of Haemophilus ducreyi lacks the ability to produce cytopathic effects on human foreskin fibroblasts. Mol Microbiol 21:13-9.
43. Parsons, N.J., J. A. Cole, and H. Smith. 1990. Resistance to human serum of B gonococci in urethral exudates is reduced by neuraminidase. Proc R Soc Lond Biol Sci 241:3-5.
44. Phillips, N. J., C. M. John, L. G. Reinders, B. W. Gibson, M. A. Apicella, and J. M. Griffiss. 1990. Structural models for the cell surface lipooligosaccharides of Neisseria gonorrhoeae and Haemophilus influenzae. Biomed Environ Mass Spectrom 19:731-45.
45. Plumbridge, J., and E. Vimr. 1999. Convergent pathways for utilization of the amino sugars N-acetylglucosamine, N-acetylmannosamine, and N-acetylneuraminic acid by Escherichia coli. J Bacteriol 181:47-54.
46. Post, D. M., R. Mungur, B. W. Gibson, and R. S. Munson Jr. Unpublished data.
47. Preston, A., R. E. Mandrell, B. W. Gibson, and M. A. Apicella. 1996. The lipooligosaccharides of pathogenic gram-negative bacteria. Crit. Rev Microbiol 22: 139-80.
48. Ringenberg, M. A., S. M. Steenbergen, and E. R. Vimr. 2003. The first committed step in the biosynthesis of sialic acid by Escherichia coli K1 does not involve a phosphorylated N-acetylmannosamine intermediate. Mol Microbiol 50:961-75.
49. San Mateo, L. R., M. M. Hobbs, and T. H. Kawula. 1998. Periplasmic copper-zinc superoxide dismutase protects Haemophilus ducreyi from exogenous superoxide. Mol Microbiol 27:39 1-404.
50. Schilling, B., S. Goon, N. M. Samuels, S. P. Gaucher, J. A. Leary, C. R. Bertozzi, and B. W. Gibson. 2001. Biosynthesis of sialylated lipooligosaccharides in Haemophilus ducreyi is dependent on exogenous sialic acid and not mannosamine. Incorporation studies using N-acylmannosamine analogues, N-glycolylneuraminic acid, and ¹³C-labeled N-acetylneuraminic acid. Biochemistry 40:12666-77.
51. Schweda, E. K., J. A. Jonasson, and P. E. Jansson. 1995. Structural studies of lipooligosaccharides from Haemophilus ducreyi I™ 5535, ITM 3147, and a fresh clinical isolate, ACY1: evidence for intrastrain heterogeneity with the production of mutually exclusive sialylated or elongated glycoforms. J Bacteriol 177:5316-21.
52. Schweda, E. K., A. C. Sundstrom, L. M. Eriksson, J. A. Jonasson, and A. A. Lindberg. 1994. Structural studies of the cell envelope lipopolysaccharides from Haemophilus ducreyi strains ITM 2665 and ITM 4747. J Biol Chem 269:12040-8.
53. Spinola, S. M., A. Orazi, J. N. Arno, K. Fortney, P. Kotylo, C. Y. Chen, A. A. Campagnari, and A. F. Hood. 1996. Haemophilus ducreyi elicits a cutaneous infiltrate of CD4 cells during experimental human infection. J Infect Dis 173:394-402.
54. Steen, R. 2001. Eradicating chancroid. Bull World Health Organ 79:818-26.
55. Steenbergen, S. M., C. A. Lichtensteiger, R. Caughlan, J. Garfinlde, T. E. Fuller, and E. R. Vimr. 2005. Sialic Acid metabolism and systemic pasteurellosis. Infect Immun 73:1284-94.
56. Stevens, M. K., S. Porcella, J. Klesney-Tait, S. Lumbley, S. E. Thomas, M. V. Norgard, J. D. Radolf, and E. J. Hansen. 1996. A hemoglobin-binding outer membrane protein is involved in virulence expression by Haemophilus ducreyi in an animal model. Infect Immun 64:1724-35.
57. Sun, S., N. K. Scheffler, B. W. Gibson, J. Wang, and R. S. Munson, Jr. 2002. Identification and characterization of the N-acetylglucosamine glycosyltransferase gene of Haemophilus ducreyi. Infect Immun 70:5887-92.
58. Swords, W. E., B. A. Buscher, K. Ver Steeg II, A. Preston, W. A. Nichols, J. N. Weiser, B. W. Gibson, and M. A. Apicella. 2000. Non-typeable Haemophilus influenzae adhere to and invade human bronchial epithelial cells via an interaction of lipooligosaccharide with the PAF receptor. Mol Microbiol 37:13-27.
59. Swords, W. E., M. R. Ketterer, J. Shao, C. A. Campbell, J. N. Weiser, and M. A. Apicella. 2001. Binding of the non-typeable Haemophilus influenzae lipooligosaccharide to the PAF receptor initiates host cell signalling. Cell Microbiol 3:525-36.
60. Swords, W. E., M. L. Moore, L. Godzicki, G. Bukofzer, M. J. Mitten, and J. VonCannon. 2004. Sialylation of lipooligosaccharides promotes biofilm formation by nontypeable Haemophilus influenzae. Infect Immun 72:106-13.
61. Telzak, E. E., M. A. Chiasson, P. J. Bevier, R. L. Stonebumer, K. G. Castro, and H. W. Jaffe. 1993. HV-1 seroconversion in patients with and without genital ulcer disease. A prospective study. Ann Intern Med 119:1181-6.
62. Trees, D. L., and S. A. Morse. 1995. Chancroid and Haemophilus ducreyi: an update. Clin Microbiol Rev 8:357-75.
63. Tsai, C. M. 2001. Molecular mimicry of host structures by lipooligosaccharides of Neisseria meningitidis: characterization of sialylated and nonsialylated lacto-N-neotetraose (Galâ1-4GlcNAcâ1-3Galâ1-4Glc) structures in lipooligosaccharides using monoclonal antibodies and specific lectins. Adv Exp Med Biol 491:525-42.
64. Tsai, C. M., and C. E. Frasch. 1982. A sensitive silver stain for detecting lipopolysaccharides in polyacrylamide gels. Anal Biochem 119:115-9.
65. Unkmeir, A., U. Kammerer, A. Stade, C. Hubner, S. Haller, A. Kolb-Maurer, M. Frosch, and G. Dietrich. 2002. Lipooligosaccharide and polysaccharide capsule: virulence factors of Neisseria meningitidis that determine meningococcal interaction with human dendritic cells. Infect Immun 70:2454-62.
66. Vimr, E., C. Lichtensteiger, and S. Steenbergen. 2000. Sialic acid metabolism's dual function in Haemophilus influenzae. Mol Microbiol 36:1113-23.
67. Vimr, E. R., K. A. Kalivoda, E. L. Deszo, and S. M. Steenbergen. 2004. Diversity of microbial sialic acid metabolism. Microbiol. Mol Biol Rev 68:132-53, table of contents.
68. Vimr, E. R., and F. A. Troy. 1985. Identification of an inducible catabolic system for sialic acids (nan) in Escherichia coli. J Bacteriol 164:845-53.
69. Vimr, E. R., and F. A. Troy. 1985. Regulation of sialic acid metabolism in Escherichia coli: role of N-acylneuraminate pyruvate-lyase. J Bacteriol 164:854-60.
70. Vogel, U., A. Weinberger, R. Frank, A. Muller, J. Kohl, J. P. Atkinson, and M. Frosch. 1997. Complement factor C3 deposition and serum resistance in β isogenic capsule and lipooligosaccharide sialic acid mutants of serogroup Neisseria meningitidis. Infect Immun 65:4022-9.
71. Wood, G. E., S. M. Dutro, and P. A. Totten. 1999. Target cell range of Haemophilus ducreyi hemolysin and its involvement in invasion of human epithelial cells. Infect Immun 67:3740-9.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of inhibiting the growth and/or proliferation and/or infectivity of a bacterium, said method comprising contacting said bacterium with an inhibitor of an ABC transporter of sialic acid.

2. A method of treating a mammal infected with a bacterium, said method comprising administering an inhibitor of a bacterial ABC transporter of sialic acid, wherein the inhibitor is administered in an amount that reduces the uptake of sialic acid by the bacterium.

3. The method of claim 1, wherein the bacterium is a pathogenic organism containing an ABC transporter that transports sialic acid wherein said ABC transporter has high homology to the ABC transporter comprising SatA, SatB, SatC, and SatD proteins, and has sialic acid as a part of its cell wall structure.

4. The method of claim 3, wherein the bacterium is a member of the Pasteurellaceae family.

5. The method of claim 3, wherein the bacterium is a gram-positive bacterium.

6. The method of claim 3, wherein the bacterium is selected from the group consisting of Haemophilus ducreyi, Haemophilus influenzae, Haemophilus somnus, Haemophilus gallarium, Vibrio vulnificus, Vibrio cholera, Shigella flexneri, Pseudomonas aeruginosa, Helicobacter pylori, Pasturella multicidia, Salmonella enteritidis, Actinobacillus pleuropneumoniae. Haemophilus somnus, Corynebacterium glutamicum, Corynebacterium diphtheriae, and Streptomyces avermitilis.

7. The method of claim 3, wherein the bacterium is Haemophilus ducreyi.

8. The method of claim 3, wherein the inhibitory agent is a viral neuraminidase inhibitor.

9. The method of claim 8, wherein the viral neuraminidase inhibitor is selected from the group consisting of 3-fluoro-N-acetylneuarmainic acid (3FNA), N-acetyl-2,3-didehydro-2-deoxyneuraminic acid (DDNA), the 4-guanidino-derivative of DDNA (Relenza®), and Tamiflu®.

10. The method of claim 9, wherein the inhibitory agent is an N-alkanoyl-derivative of sialic acid.

11. The method of claim 10, wherein the N-alkanoyl-derivative of sialic acid is a 5-N-octanoyl derivative of sialic acid (SiaOct).

12. The method of claim 3, wherein the uptake of sialic acid by the bacterium is reduced by 50%.

13. The method of claim 3, wherein the mammal is diagnosed with a pathology selected from the group consisting of otitis media, otitis media with effusion, pneumonia, chronic bronchitis, and chancroid.

14. An isolated nucleic acid the encodes one or more proteins selected from the group consisting of SatA (SEQ ID NO:5), SatB (SEQ ID NO: 6), SatC (SEQ ID NO: 7), and SatD (SEQ ID NO: 8).

15. The nucleic acid of claim 14, wherein said nucleic acid is present in a vector.

16. A host cell comprising a heterologous nucleic acid that encodes one or more proteins selected from the group consisting of SatA (SEQ ID NO:5), SatB (SEQ ID NO: 6), SatC (SEQ ID NO: 7), and SatD (SEQ ID NO: 8).

17. The host cell of claim 16, wherein said nucleic acid comprises one or more sequences selected from the group consisting of SEQ ID NO:1, SEQ ID NO:2, SEQ ID NO:3, and SEQ ID NO:4.

18. The host cell of claim 16, wherein said cell is a prokaryotic cell.

19. The host cell of claim 16, wherein said cell is a bacterial cell.

20. The host cell of claim 16, wherein said cell is a eukaryotic cell.

21. An isolated peptide comprising a sialic acid ABC transporter.

22. The isolated peptide of claim 21, wherein said peptide is selected from the group consisting of SatA (SEQ ID NO:5), SatB (SEQ ID NO: 6), SatC (SEQ ID NO: 7), and SatD (SEQ ID NO: 8).

23. An antibody that specifically binds a protein that is a component of a sialic acid ABC transporter.

24. The antibody of claim 23, wherein said antibody specifically binds a protein comprising at least 10 contiguous amino acids of a protein selected form the group consisting of SatA (SEQ ID NO:5), SatB (SEQ ID NO: 6), SatC (SEQ ID NO: 7), and SatD (SEQ ID NO: 8).

25. The antibody of claim 23, wherein said antibody specifically binds a protein selected form the group consisting of SatA (SEQ ID NO:5), SatB (SEQ ID NO: 6), SatC (SEQ ID NO: 7), and SatD (SEQ ID NO: 8).

26. A method of screening for an agent that inhibits the growth, proliferation, or infectivity of a bacterium, said method comprising:
contacting a sialic acid ABC transporter with a test agent; and identifying agents that bind to said ABC transporter, where an agent that binds to said ABC transporter is a putative agent that inhibits the growth, proliferation, or infectivity of a bacterium.

27. The method of claim 26, wherein said transporter comprises a protein having at least 60% sequence identity with a protein selected from the group consisting of SatA (SEQ ID NO:5), SatB (SEQ ID NO: 6), SatC (SEQ ID NO: 7), and SatD (SEQ ID NO: 8).

28. The method of claim 26, wherein said transporter comprises a protein selected from the group consisting of SatA (SEQ ID NO:5), SatB (SEQ ID NO: 6), SatC (SEQ ID NO: 7), and SatD (SEQ ID NO: 8).

29. A method of screening for an agent that inhibits the growth, proliferation, or infectivity of a bacterium, said method comprising:
screening a test agent for the ability to inhibit the expression and/or activity of a sialic acid ABC transporter, where an agent that inhibits the expression nor activity of the sialic acid ABC transporter is a putative agent that inhibits the growth, proliferation, or infectivity of a bacterium.

30. The method of claim 29, wherein said transporter comprises a protein having at least 60% sequence identity with a protein selected from the group consisting of SatA (SEQ ID NO:5), SatB (SEQ ID NO: 6), SatC (SEQ ID NO: 7), and SatD (SEQ ID NO: 8).

31. The method of claim 29, wherein said transporter comprises a protein selected from the group consisting of SatA (SEQ ID NO:5), SatB (SEQ ID NO: 6), SatC (SEQ ID NO: 7), and SatD (SEQ ID NO: 8).

32. A method of treating a patient against bacterial colonization or infection comprising administering to the patient an effective amount of a sialic acid ABC transporter inhibitory agent.

33. The method of claim 32, wherein said agent is present in a physiologically acceptable excipient.

34. The method of claim 32, wherein said agent is formulated as a unit dosage formulation.

35. The method of claim 32, wherein said bacterial colonization or infection is colonization or infection by a bacterium that is a pathogenic organism containing an ABC transporter that transports sialic acid wherein said ABC transporter has high homology to the ABC transporter comprising SatA, SatB, SatC, and SatD proteins, and has sialic acid as a part of its cell wall structure.

36. The method of claim 32, wherein said bacterial colonization or infection is colonization or infection by a bacterium that is a member of the Pasteurellaceae family.

37. The method of claim 32, wherein said bacterial colonization or infection is colonization or infection by a bacterium that is a gram-positive bacterium.

38. The method of claim 32, wherein said bacterial colonization or infection is colonization or infection by a bacterium selected from the group consisting of Haemophilus ducreyi, Haemophilus influenzae, Haemophilus somnus, Haemophilus gallarium, Vibrio vulnificus, Vibrio cholera, Shigella flexneri, Pseudomonas aeruginosa, Helicobacter pylori, Pasturella multicidia, Salmonella enteritidis, Actinobacillus pleuropneumoniae. Haemophilus somnus, Corynebacterium glutamicum, Corynebacterium diphtheriae, and Streptomyces avermitilis.

39. The method of claim 32, wherein said bacterial colonization or infection is colonization or infection by Haemophilus ducreyi.


SEQUENCE LISTING (8 sequences)

SEQ ID NO: 1 - DNA, 1578 bp, Haemophilus ducreyi
atgacttctcattttgaatacaatcaatctcgccgccattttatgaaattactttcaagtgctggcgttggtattgcattcagtggcacgctaggtgcattttcagccgatgcgtttagctcgccttcaggctcaacaattgaagcagggattgcatatccaatttcaaccggttttgatcctttaacctcaagtggtgcatcgtcaatggcagccaatctgcatattttcgaaggtttagtggatttacatccggtaactcgtcagccttatttggcactagccgctaaagaacctgagcaaaaagatgatttaacttattatattagcttacgggaaggcgcaatgttccacgacggtagccctgttactactgaagatgtagtctattcatttgaacgcgttttagatccggcaaaagcctcactatttgctcaatttattccctttattgcttctgttacagcactagatgacaatgttgttgaattcaaattgaaatacccttttgctttattcaaagagcggttaacaattattaaaattgtacccaaacatattgtcgaagctggacaatctgcttttgatgctaaacctatcggttccggtccatacaaatttgtttctgcgacaaaagatgatcgcatcgtatttgaagcaaataccgtatataacgggcactatcctgctaaagtagaaaagatgacttggttcttattatctgacgatgcagcacgtgttacagctcaagaatcaggtcgtgtgcaagcgattgaatctgtgccgtatcttgatgctgaacgcttaaaacgtaaaaataatgttgagtctgtacaatcatttggcctgctctttttaatgtttaattgcgaaaaagcaccatttgataaccctaaagtgcgccaagcattacattatgctttagataagcaaaaattaattgatattgttttcttagggaacgctaaagcagcaacgtcttacctacaggatacccacccagattatgtcaaagcgagttcacaatatgattatgataaagctaaagcggaaaaattattagctgaagcaggaatcaccaacttaactttccaattactcgcaactgatcatgcttgggttaaagaatgtgcgccattaattctagagtcttggaatgcgctgagtgtggtaaaagtcacgctacaacatttacaatcaggtgcgttatatagtgcgcatgtcgataaaggtgcttatgaagtcgttattgcacccggagatccgtccgtatttggcaacgatttagacttattattaagttggtggtatcgtggtgatgtttggccaaaacgccgcttccgttgggcaaataccgcagaatatcacgaagtacaaaaattactggacgaagcaataaaaaatcctgcaggctcaaaagttgcttggcaaaaagccatcaatattattgctgaacaggtgccactttatcccatcatacaccgtaaattaccaactgcgtggaatactaaaaaactaaccgatttccaaccattaccgacaacaggtttgtctttccttggtgtaggtcgtacataa

SEQ ID NO: 2 - DNA, 954 bp, Haemophilus ducreyi
atgcaaatcatactccgattattatggcggcgtttaatggctcttcccataatgatcttaggtgttaccgcccttgtatttatcgtattacaattcacgcccggtgatcccgccactgttgcgctaggagaaagtgcttcggaagtcgctaaagaaatttatcgacaacaacacggcttaaatgatcctttggttgtgcaatatgttcgttttttagctaatctagtggtacttgatttttggctaactacaccgccacaacagccgattattgatatgatcgcaaaagcatttccactcaccttacaactgacattgattggcgtatttttagccgctaccgtctctttttctctcggtattttggctgccctttatcgtgatcgttggattgatcaaattatccgcttaatttctgtcgcagcagttgcaacgccttctttttggctaggtatcttactcattcaatatttttccttaacacttgattggctaccttcaggtggttttgtaccgcttagtgaagatccacatgaatacgcccgatcaatgattctgccatcacttgctttagctttacctgtttgtgcctcattgattcgtgtcgtacgaactacgatggtagaagaaatggataaagattatgtaagaaccgctatcggtaatggcgtgccttatgcaactgtcattcggcataatgtattacgcaatgcgcttattacccccgtaacgatattaggtttacgggttggctatttacttggaggagcggtcgttattgaacaaatttttgatctaccgggtatgggaaaattaattttcaatggcattgtcaatcatgatttacatttagtgcaaggcgtagttttaaccatcgcattcacctttgttttagtcaatattttggtagatattttatacttatttatcaaccctaaaatccggagtctataa

SEQ ID NO: 3 - DNA, 1956 bp, Haemophilus ducreyi
atgtttcgtcaagcattagcgcatagacttgcgtcaggcagtgcaaggcttaaatcattacccaccagttcaaaaatcgccttgatatttatgctattttttgccgttattgccattttagcccccatagttgcgccttataatcctttacaaacactgagaccggttcaagcaccgagtacagactacttatttggtacagaccgcctaggtagagatattttctcgcggatactatggggcgcaagaacatcgctctttattggtttaggtgcggttgcttttgctattctattgggcggtgtattaggtgcaactgctgcaaccgctgatagacttggcaatgaagtgattatgcgatcaatggatatcttaatggcatttccaggtatcgcattagctgccgtattactcgcaacttttggtaattctgtaccggttattattattaccatagcggtcgtttatacgccacaacttgcacgtgttgtacgtgctaatgtcgtagcacaatgggaggaagattatgttcgtgctgaacgagtattaggaggtagccgtacttatattctcgtaaaacacgtagtacgtaataccgctgcgcctgtattcgtttttgccaccgttatggttgccgatgcaattgtttttgaagcttcgctctctttcttaggggcgggagtacagccacctttcccatcgtggggaaatattttagcggaaggccgtaatttggtgttaagtggtttttggtgggcaacgacttttgcaggcctgatgattctactcacggtactcgcattaaatatcttagcagaaggcttaactgatgccttagtcaatccaaaactaaaacgctcaaccaaaaataaagacgatgtgactaaaccacttgctactgatgtacaagaagcaatggcagaaacactcgcgttaaaacgctatttactgcgtttgcacgaaaaagaaatgtctcggacagacagaatgatattaaatccaaacacaaacgtaattttacaggttgagaatttatccattcgttttcccaatcgttacaatgaaataccgttagttgataatattagttttactattcatgaaggcgaaacaatgggattagtcggcgaatcaggctgtggcaaatctatcacggcgttttcgattatgggattattacctcaaacagcgaaaattaccggtaaaattttatttactgatcggagtggcaaacaacacgatttattgaaatcagctaatttaaatgcgctacgtggccacgaaatttcgatgatttatcaagatgcactcagtgcattaaatccttcaatgaagattaaagatcaaatggcacaattgattagcctaggcggtaaacactcttctgaaacgttattacattgggtaaaactggatccagctaaaaccttaaatcgctatccacacgaactttctggtggccaacgacaacgtgtattaattgctatggcattagctagagaacctaagctattgattgcagatgaaccgacgaccgctttagatgtaacggtacaagctgaagttatcaagctattaaatgaattacgtgaaaaactcggttttgctatggtgtttgttagccacgatttagcattagttgcgcaaattgcacatcacattaccgtaatgtatgccggtcaagtggtggaaaaagcacctactacgccattgcttgctcatccaacccacgaatatactcgtggcttacttggttcggtcctttcaacggaagtgcgtgccaagcgtttatatcaaatcccggggagtgttccatcgccgtttgattttgctaaaggagaccgctttgctagccgttcattacgcccaaatgccgatccaaaccaacgattaacgatggtagcgactgcggatcatcctgaacatttttgggcctctcacttagagaaaaattaa

SEQ ID NO: 4 - DNA, 807 bp, Haemophilus ducreyi
atgagcataaaagtagtaaaagaaaagccaattattgaattatctaatattgttgtacagtttacttctcgtgatagaagtttgtttaatcccaagaaatttaccgcagtagataatgtcaatttagccattcatgccggtgaaacagtgggcttggttggtcaatcgggttgtggtaaatcaactttagcaaatattatgatcggtttacaaaaaccaacctccggctctgtgaaattcaatggtttagaaatgcggtataactgtgccgaagcgcgcaaatattttggtagccaagtttcggttatcttccaagatccctcaacggcactaaatcctcgtatgcgggtattaaatatcttaaaagatcctatggatatacataatattctgctaccacaagaacgtgaaaaacgcgtttatcaattactttcaagagtcggtttacctcgctctgtagcattagttgagccaacacgtttatcaggcgggcaaaaacagcgggtggcaattgccagagcattagcacttaatccaaaattgatcgtagcagatgaaccaacttctgcgttagatgtatccgtacgtgctcaagtattgaatctacttgctgatttaaaaaaagaattaaatttggcaatgctgttcatttctcatgatttacaaacagttcatcaagtatctgaccgcattgttgtaatgaatggcggtaaagtagtcgaaacaggccgtgccgatcaagtatttaatcagcctcgtgaagaatatacacgtacattacttaatgcaacaccgtccttgttataa

SEQ ID NO: 5 - protein, 525 aa, Haemophilus ducreyi
MTSHFEYNQSRRHFMKLLSSAGVGIAFSGTLGAFSADAFSSPSGSTIEAGIAYPISTGFDPLTSSGASSMAANLHIFEGLVDLHPVTRQPYLALAAKEPEQKDDLTYYISLREGAMFHDGSPVTTEDVVYSFERVLDPAKASLFAQFIPFIASVTALDDNVVEFKLKYPFALFKERLTIIKIVPKHIVEAGQSAFDAKPIGSGPYKFVSATKDDRIVFEANTVYNGHYPAKVEKMTWFLLSDDAARVTAQESGRVQAIESVPYLDAERLKRKNNVESVQSFGLLFLMFNCEKAPFDNPKVRQALHYALDKQKLIDIVFLGNAKAATSYLQDTHPDYVKASSQYDYDKAKAEKLLAEAGITNLTFQLLATDHAWVKECAPLILESWNALSVVKVTLQHLQSGALYSAHVDKGAYEVVIAPGDPSVFGNDLDLLLSWWYRGDVWPKRRFRWANTAEYHEVQKLLDEAIKNPAGSKVAWQKAINIIAEQVPLYPIIHRKLPTAWNTKKLTDFQPLPTTGLSFLGVGRT

SEQ ID NO: 6 - protein, 317 aa, Haemophilus ducreyi
MQIILRLLWRRLMALPIMILGVTALVFIVLQFTPGDPATVALGESASEVAKEIYRQQHGLNDPLVVQYVRFLANLVVLDFWLTTPPQQPIIDMIAKAFPLTLQLTLIGVFLAATVSFSLGILAALYRDRWIDQIIRLISVAAVATPSFWLGILLIQYFSLTLDWLPSGGFVPLSEDPHEYARSMILPSLALALPVCASLIRVVRTTMVEEMDKDYVRTAIGNGVPYATVIRHNVLRNALITPVTILGLRVGYLLGGAVVIEQIFDLPGMGKLIFNGIVNHDLHLVQGVVLTIAFTFVLVNILVDILYLFINPKIRSL

SEQ ID NO: 7 - protein, 651 aa, Haemophilus ducreyi
MFRQALAHRLASGSARLKSLPTSSKIALIFMLFFAVIAILAPIVAPYNPLQTLRPVQAPSTDYLFGTDRLGRDIFSRILWGARTSLFIGLGAVAFAILLGGVLGATAATADRLGNEVIMRSMDILMAFPGIALAAVLLATFGNSVPVIIITIAVVYTPQLARVVRANVVAQWEEDYVRAERVLGGSRTYILVKHVVRNTAAPVFVFATVMVADAIVFEASLSFLGAGVQPPFPSWGNILAEGRNLVLSGFWWATTFAGLMILLTVLALNILAEGLTDALVNPKLKRSTKNKDDVTKPLATDVQEAMAETLALKRYLLRLHEKEMSRTDRMILNPNTNVILQVENLSIRFPNRYNEIPLVDNISFTIHEGETMGLVGESGCGKSITAFSIMGLLPQTAKITGKILFTDRSGKQHDLLKSANLNALRGHEISMIYQDALSALNPSMKIKDQMAQLISLGGKHSSETLLHWVKLDPAKTLNRYPHELSGGQRQRVLIAMALAREPKLLIADEPTTALDVTVQAEVIKLLNELREKLGFAMVFVSHDLALVAQIAHHITVMYAGQVVEKAPTTPLLAHPTHEYTRGLLGSVLSTEVRAKRLYQIPGSVPSPFDFAKGDRFASRSLRPNADPNQRLTMVATADHPEHFWASHLEKN

SEQ ID NO: 8 - protein, 268 aa, Haemophilus ducreyi
MSIKVVKEKPIIELSNIVVQFTSRDRSLFNPKKFTAVDNVNLAIHAGETVGLVGQSGCGKSTLANIMIGLQKPTSGSVKFNGLEMRYNCAEARKYFGSQVSVIFQDPSTALNPRMRVLNILKDPMDIHNILLPQEREKRVYQLLSRVGLPRSVALVEPTRLSGGQKQRVAIARALALNPKLIVADEPTSALDVSVRAQVLNLLADLKKELNLAMLFISHDLQTVHQVSDRIVVMNGGKVVETGRADQVFNQPREEYTRTLLNATPSLL